(19) United States
(12) Patent Application Publication
(10) Pub. No.: US 2013/0327251 A1
(43) Pub. Date: Dec. 12, 2013

(54) STABLE HOMOGENEOUSLY MIXED NANOSCALE COATINGS DERIVED FROM UNIQUE MULTI-FUNCTIONAL AND MULTIDENTATE AROMATIC ADSORBATES

(71) Applicant: THE UNIVERSITY OF HOUSTON SYSTEM, Houston, TX (US)

(72) Inventors: T. Randall Lee, Houston, TX (US); Supachai Rittikulsittichai, Houston, TX (US); Thomas Frank, Ausint, TX (US); Burapol Singhana, Phon Sai (TH)

(73) Assignee: THE UNIVERSITY OF HOUSTON SYSTEM, Houston, TX (US)

(21) Appl. No.: 13/788,974

(22) Filed: Mar. 7, 2013

Related U.S. Application Data
(60) Provisional application No. 61/607,710, filed on Mar. 7, 2012.

(51) Int. Cl. C07C 323/20; C09D 5/00; C07C 319/02; C07D 213/32

(52) U.S. Cl. CPC C07C 323/20; C07D 213/32; C09D 5/00; C07C 319/02
USPC 106/287.2; 568/62; 546/339; 546/291; 546/296; 546/297; 546/301; 546/302; 546/290; 546/304; 546/307; 546/309; 546/312; 546/308; 546/323; 546/316; 106/287.23; 106/287.25; 106/287.28; 106/287.3; 106/287.32

(57) ABSTRACT

Novel tridentate-, bidentate-, and monodentate-based aromatic adsorbates including self-assembled monolayers (SAMs), especially, mixed multi-component SAMs, where the adsorbates comprise an aromatic ring including one head group or a plurality of dentate head groups and one tunable tail group or a plurality of tail groups and methods for making the same, and methods for using same, their use in the preparation of homogeneously mixed multi-component self-assembled monolayers (SAMs). The adsorbants and SAMs derived therefrom are ideally suited for biosensing, biosensing diagnostics, biological interfacial mimics, surface protections for nanoparticles, inert coatings for artificial implants, and corrosion-resistant coatings for microelectronics components.

### RELATED APPLICATIONS

This application claims the benefit to and priority to U.S. Provisional Application Ser. No. 61/607,710 filed 7 Mar. 2012 (Mar. 7, 2012).

### GOVERNMENTAL SPONSORSHIP

Not Applicable.

### REFERENCE TO A SEQUENTIAL LISTING

Not Applicable.

### BACKGROUND OF THE INVENTION

1. Field of the Invention

Embodiments of the present invention relate to the compositions of new tridentate-, bidentate-, and monodentate-based aromatic adsorbates, methods for making the same, and methods for their use in the preparation of self-assembled monolayer and especially homogeneously mixed multi-component self-assembled monolayers (SAMs). Embodiments of the compositions exhibit homogenous lateral chain distributions, no phase separation or islanding across the surfaces, and enhanced stability as compared to standard monodentate adsorbates. Embodiments of the compositions of this invention are well suited for such applications as, without limitation, biosensing, biosensing diagnostics, biological interfacial mimics, surface protections for nanoparticles, inert coatings for artificial implants, and corrosion-resistant coatings for microelectronics components.

2. Description of the Related Art

Ultrathin organic coatings have become a promising strategy for modern material science, especially, a method of surface functionalization, because they offer a wide range of chemical functionalities, which can be adjusted and tailored via organic synthetic transformations to make chemical compositions having specific tail groups to fabricate custom-designed SAMs for specific applications. The successful use of these modified surface materials for most of practical applications (i.e., nanofabrication,¹ microelectronic devices,^{2,3} and mechanical interfacial applications involving friction,^{4,5} corrosion,^{6,7} lubrication, and adhesion^{8,9}) critically relies on their stabilities. Furthermore, several applications (e.g., platforms for chemical and biological sensors,^{10,11} artificial implants^{12,13}) require complex mixed surface functionalities, where the lateral distribution of chemical functionalities are homogeneous across surfaces and can be precisely controlled at the nano-scales. Numerous approaches have been utilized for the preparation of mixed SAM coatings, including the use of unsymmetrical dialkyl sulfides or dialkyl sulfides,^{14,15} the use of two or more different adsorbates¹⁶, and the use of ligand-exchange methods.¹⁷ However, SAM coatings derived by these strategies are critically limited due to the difficulty in controlling the distribution and the ratios of the two (or more) adsorbates. Moreover, several studies have found that these types of SAMs are unstable and suffer from phase separation (i.e., the formation of segregated domains or “islands” of each adsorbate). While other fabrication techniques such as microcontact printing and lithographic methods have been developed to pattern multicomponent SAMs on surfaces,¹ most of these techniques are costly and suffer from degradation due to molecular desorption and/or diffusion.

Thus, there is a need in the art for compositions capable of producing stable mixed SAMs, for methods of preparing the compositions, SAM modified substrates, and apparatus incorporating the SAM modified substrates.

### SUMMARY OF THE INVENTION

As an improvement over disadvantages of aforementioned strategies, embodiments of the present invention provide novel and convenient methods for preparing SAMs and especially homogeneously mixed multi-component SAMs via the adsorption of new tridentate-, bidentate-, and monodentate-based aromatic adsorbates non-symmetrical spiroalkanedithiols onto appropriate surfaces including metal surfaces.^{18,19} Embodiments of the present invention also provide that mixed SAMs derived from this class of bidentate adsorbate exhibit homogenous lateral chain distributions, no phase separation or islanding across SAM modified surfaces, and enhanced stability as compared to standard monodentate adsorbates. Embodiments of the present invention also provide absorbates having three different tail groups.

Embodiments of the present invention provide mono-, bi-, and tri-dentate absorbates for the preparation of self-assembled monolayers (SAMs), especially, mixed multi-component SAMs, where the adsorbates comprise an aromatic ring including one head group or a plurality of dentate head groups and one tunable tail group or a plurality of tail groups designated, Z¹, Z², and Z³ groups. Embodiments for these adsorbates comprise compounds of the general formula (I):

Z¹,Z²,Z³-A(SH)ₖ(RSH)ₗ(CHₘ₁(SH)ₘ₂)ₙ (I)

where: (1) A is an aromatic group, where one of the carbon atoms of the group may be replaced by an oxygen atom (O), a nitrogen atom (N), or a sulfur atom (S), (2) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹, Z², or Z³ groups is not a hydrogen atom, (3) R is the same or different carbyl groups having between 1 and 3 carbon atoms, (4) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (5) the R^{a2-a4} may be linear or branched carbyl groups having between about 12 to 40 carbon atoms, (6) the R^{d,f,x} groups may be hydrogen atoms or linear or branched carbyl groups having between 1 and 10 carbon atoms, (7) R^{g,h,i,w,z} are linear or branched carbyl groups having between 1 and 10 carbon atoms, (8) k, l, m1, m2 and n are integers, (9) k has a value between 1 and 3, when l and n have a value of 0, (10) l has a value between 1 and 3, when k and n have a value of 0, (11) n has a value of 1, when k and l have a value of 0, and (12) m1+m2 is equal to 3. In certain embodiments, at least two of Z¹, Z², and Z³ groups are not hydrogen atoms. In other embodiments, all of the Z¹, Z², and Z³ groups are not hydrogen atoms.

Embodiments of the present invention provide mono-, bi- and tri-dentate absorbates for the preparation of self-assembled monolayers (SAMs), especially, mixed multi-component SAMs, where the adsorbates comprise compounds of the general formula (II):

Z¹,Z²,Z³-ACHₘ₁(SH)ₘ₂ (II)

where: (1) A is an aromatic group, where one of the carbon atoms of the group may be replaced by an oxygen atom (O), a nitrogen atom (N), or a sulfur atom (S), (2) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹, Z², or Z³ groups is not a hydrogen atom, (3) R is the same or different carbyl groups having between 1 and 3 carbon atoms, (4) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (5) the R^{a2-a4} may be linear or branched carbyl groups having between about 12 to 40 carbon atoms, (6) the R^{d,f,x} groups may be hydrogen atoms or linear or branched carbyl groups having between 1 and 10 carbon atoms, (7) R^{g,h,i,w,z} are linear or branched carbyl groups having between 1 and 10 carbon atoms, (8) m1 and m2 are integers, and (9) m1+m2 is equal to 3. In certain embodiments, at least two of Z¹, Z², and Z³ groups are not hydrogen atoms. In other embodiments, all of the Z¹, Z², and Z³ groups are not hydrogen atoms.

Embodiments of the present invention provide mono-, bi- and tri-dentate absorbates for the preparation of self-assembled monolayers (SAMs), especially, mixed multi-component SAMs, where the adsorbates comprise compounds of the general formula (III):

Z¹,Z²,Z³-A-m-(RSH)₂ (III)

where: (1) A is an aromatic group, where one of the carbon atoms of the group may be replaced by an oxygen atom (O), a nitrogen atom (N), or a sulfur atom (S), (2) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹, Z², or Z³ groups is not a hydrogen atom, (3) R is the same or different carbyl groups having between 1 and 3 carbon atoms, (4) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (5) the R^{a2-a4} may be linear or branched carbyl groups having between about 12 to 40 carbon atoms, (6) the R^{d,f,x} groups may be hydrogen atoms or linear or branched carbyl groups having between 1 and 10 carbon atoms, and (7) R^{g,h,i,w,z} are linear or branched carbyl groups having between 1 and 10 carbon atoms. In certain embodiments, at least two of Z¹, Z², and Z³ groups are not hydrogen atoms. In other embodiments, all of the Z¹, Z², and Z³ groups are not hydrogen atoms.

Embodiments of the present invention provide mono-, bi- and tri-dentate absorbates for the preparation of self-assembled monolayers (SAMs), especially, mixed multi-component SAMs, where the adsorbates comprise compounds of the general formula (IV):

Z¹,Z²,Z³-Py-m-(RSH)₂ (IV)

where: (1) Py is pyridine, (2) -m- means the two RSH groups meta to each other, (3) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹, Z², and Z³ groups is not hydrogen, (4) R is the same or different carbyl groups having between 1 and 3 carbon atoms, (5) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (6) the R^{a2-a4} may be linear or branched carbyl groups having between about 12 to 40 carbon atoms, (7) the R^{d,f,x} groups may be hydrogen atoms or linear or branched carbyl groups having between 1 and 10 carbon atoms, and (8) R^{g,h,i,w,z} are linear or branched carbyl groups having between 1 and 10 carbon atoms. In certain embodiments, at least two of the Z¹, Z² and Z³ groups are not a hydrogen atom. In other embodiments, all of the Z¹, Z², and Z³ groups are not a hydrogen atom.

Embodiments of the present invention provide mono-, bi- and tri-dentate absorbates for the preparation of self-assembled monolayers (SAMs), especially, mixed multi-component SAMs, where the adsorbates comprise compounds of the general formula (V):

Z¹,Z²-Cp-m-(RSH)₂ (V)

where: (1) Cp is 5-member aromatic ring, where one of the carbon atoms of the Cp ring may be replaced by an oxygen atom, a nitrogen atom, or a sulfur atom, (2) -m- means that the two RSH groups are meta to each other or occupy the 2 and 5 positions of the five membered Cp ring, (3) Z¹ and Z² groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹ and Z² groups is not a hydrogen atom, (4) R is the same or different carbyl groups having between 1 and 3 carbon atoms, (5) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (6) the R^{a2-a4} may be linear or branched carbyl groups having between about 12 to 40 carbon atoms, (7) the R^{d,f,x} groups may be hydrogen atoms or linear or branched carbyl groups having between 1 and 10 carbon atoms, and (8) R^{g,h,i,w,z} are linear or branched carbyl groups having between 1 and 10 carbon atoms. In certain embodiments, at least two of the Z¹, Z² and Z³ groups are not a hydrogen atom. In other embodiments, all of the Z¹, Z², and Z³ groups are not a hydrogen atom.

Embodiments of the present invention provide SAMs comprising two or more compounds set forth above.

Embodiments of the present invention provide substrates having SAMs comprising two or more compounds set forth above.

Embodiments of the present invention provide methods for preparing the mono-, bi- and tri-dentate absorbates of this invention.

Embodiments of the present invention provide methods for preparing SAMs comprising two or more compounds set forth above.

Embodiments of the present invention provide methods for preparing substrates including SAMs comprising two or more compounds set forth above.

### BRIEF DESCRIPTION OF THE DRAWINGS

The invention can be better understood with reference to the following detailed description together with the appended illustrative drawings in which like elements are numbered the same:

FIG. 1 depicts XPS spectra showing the S 2p region for monolayer films derived from C18 thiol absorbate and new classes of multidentate aromatic thiols.

FIG. 2 depicts PM-IRRAS spectra showing the C—H stretching region for monolayer films derived from C18 thiol absorbate and new classes of multidentate aromatic adsorbates.

FIG. 3 depicts Desorption profiles of SAMs derived from C18 thiol absorbate and new classes of multidentate aromatic adsorbates.

### DETAILED DESCRIPTION OF THE INVENTION

Embodiments of the present invention relates to compositions of new tridentate-, bidentate-, and monodentate-based aromatic adsorbates, methods for making the same, and methods for their use in the preparation of homogeneously mixed multi-component self-assembled monolayers (SAMs). Such systems exhibit homogenous lateral chain distributions, no phase separation (or islanding) across substrate surfaces, and enhanced stability as compared to standard monodentate adsorbates. Applications of such systems include, without limitation, biosensing, biosensing diagnostics, biological interfacial mimics, surface protections for nanoparticles, inert coatings for artificial implants, and corrosion-resistant coatings for microelectronics components.

Generally speaking, the present invention meets fundamental requirements for the use of self-assembled monolayers in research and commercial applications: (1) long-term stability of such coatings under ambient temperatures, elevated temperatures, physiological conditions, and/or sterilization conditions, and (2) facile tunability in the tail group composition and thus the surface composition, allowing the specific designs of the surfaces for certain applications.

Embodiments of the present invention provide mono-, bi-, and tri-dentate absorbates for the preparation of self-assembled monolayers (SAMs), especially, mixed multi-component SAMs, where the adsorbates comprise an aromatic ring including one head group or a plurality of dentate head groups and one tunable tail group or a plurality of tail groups designated, Z¹, Z², and Z³ groups. Embodiments for these adsorbates comprise compounds of the general formula (I):

Z¹,Z²,Z³-A(SH)ₖ(RSH)ₗ(CHₘ₁(SH)ₘ₂)ₙ (I)

where: (1) A is an aromatic group, where one of the carbon atoms of the group may be replaced by an oxygen atom (O), a nitrogen atom (N), or a sulfur atom (S), (2) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹, Z², or Z³ groups is not a hydrogen atom, (3) R is the same or different carbyl groups having between 1 and 3 carbon atoms, (4) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (5) the R^{a2-a4} may be linear or branched carbyl groups having between about 12 to 40 carbon atoms, (6) the R^{d,f,x} groups may be hydrogen atoms or linear or branched carbyl groups having between 1 and 10 carbon atoms, (7) R^{g,h,i,w,z} are linear or branched carbyl groups having between 1 and 10 carbon atoms, (8) k, l, m1, m2 and n are integers, (9) k has a value between 1 and 3, when l and n have a value of 0, (10) l has a value between 1 and 3, when k and n have a value of 0, (11) n has a value of 1, when k and I have a value of 0, and (12) m1+m2 is equal to 3. In certain embodiments, at least two of Z¹, Z², and Z³ groups are not hydrogen atoms. In other embodiments, all of the Z¹, Z², and Z³ groups are not hydrogen atoms.

The prototypical structures of the new multidentate-based aromatic adsorbates are shown below:

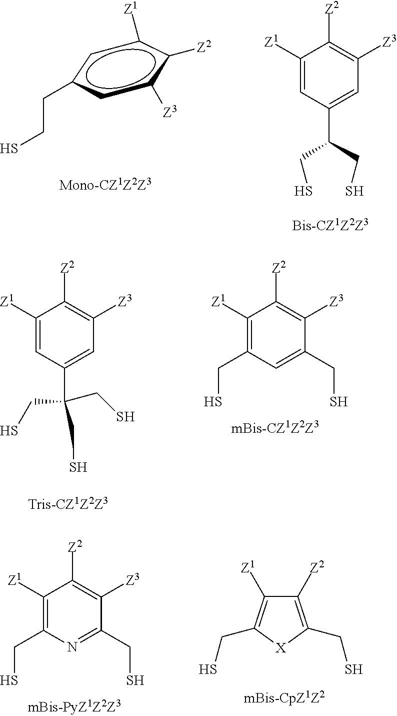

These unique adsorbates comprise an aromatic ring including one or multidentate head groups and one or tunable tail groups designated Z¹, Z², or Z³ groups.

Embodiments of the present invention provide mono-, bi- and tri-dentate absorbates for the preparation of self-assembled monolayers (SAMs), especially, mixed multi-component SAMs, where the adsorbates comprise compounds of the general formula (II):

Z¹,Z²,Z³-ACHₘ₁(SH)ₘ₂ (II)

where: (1) A is an aromatic group, where one of the carbon atoms of the group may be replaced by an oxygen atom (O), a nitrogen atom (N), or a sulfur atom (S), (2) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at t least one of the Z¹, Z², or Z³ groups is not a hydrogen atom, (3) R is the same or different carbyl groups having between 1 and 3 carbon atoms, (4) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (5) the R^{a2-a4} may be linear or branched carbyl groups having between about 12 to 40 carbon atoms, (6) the R^{d,f,x} groups may be hydrogen atoms or linear or branched carbyl groups having between 1 and 10 carbon atoms, (7) R^{g},h,i,w,z are linear or branched carbyl groups having between 1 and 10 carbon atoms, (8) m1 and m2 are integers, and (9) m1+m2 is equal to 3. In certain embodiments, at least two of Z¹, Z², and Z³ groups are not hydrogen atoms. In other embodiments, all of the Z¹, Z², and Z³ groups are not hydrogen atoms.

Embodiments of the present invention provide mono-, bi- and tri-dentate absorbates for the preparation of self-assembled monolayers (SAMs), especially, mixed multi-component SAMs, where the adsorbates comprise compounds of the general formula (III):

Z¹,Z²,Z³-A-m-(RSH)₂ (III)

where: (1) A is an aromatic group, where one of the carbon atoms of the group may be replaced by an oxygen atom (O), a nitrogen atom (N), or a sulfur atom (S), (2) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹, Z², or Z³ groups is not a hydrogen atom, (3) R is the same or different carbyl groups having between 1 and 3 carbon atoms, (4) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (5) the R^{a2-a4} may be linear or branched carbyl groups having between about 12 to 40 carbon atoms, (6) the R^{d,f,x} groups may be hydrogen atoms or linear or branched carbyl groups having between 1 and 10 carbon atoms, and (7) R^{g,h,i,w,z} are linear or branched carbyl groups having between 1 and 10 carbon atoms. In certain embodiments, at least two of Z¹, Z², and Z³ groups are not hydrogen atoms. In other embodiments, all of the Z¹, Z², and Z³ groups are not hydrogen atoms.

Embodiments of the present invention provide mono-, bi- and tri-dentate absorbates for the preparation of self-assembled monolayers (SAMs), especially, mixed multi-component SAMs, where the adsorbates comprise compounds of the general formula (IV):

Z¹,Z²,Z³-Py-m-(RSH)₂ (IV)

where: (1) Py is pyridine, (2) -m- means the two RSH groups meta to each other, (3) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹, Z², and Z³ groups is not hydrogen, (4) R is the same or different carbyl groups having between 1 and 3 carbon atoms, (5) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (6) the R^{a2-a4} may be linear or branched carbyl groups having between about 12 to 40 carbon atoms, (7) the R^{d,f,x} groups may be hydrogen atoms or linear or branched carbyl groups having between 1 and 10 carbon atoms, and (8) R^{g,h,i,w,z} are linear or branched carbyl groups having between 1 and 10 carbon atoms. In certain embodiments, at least two of the Z¹, Z² and Z³ groups are not a hydrogen atom. In other embodiments, all of the Z¹, Z², and Z³ groups are not a hydrogen atom.

Embodiments of the present invention provide mono-, bi- and tri-dentate absorbates for the preparation of self-assembled monolayers (SAMs), especially, mixed multi-component SAMs, where the adsorbates comprise compounds of the general formula (V):

Z¹,Z²-Cp-m-(RSH)₂ (V)

where: (1) Cp is 5-member aromatic ring, where one of the carbon atoms of the Cp ring may be replaced by an oxygen atom, a nitrogen atom, or a sulfur atom, (2) -m- means that the two RSH groups are meta to each other or occupy the 2 and 5 positions of the five membered Cp ring, (3) Z¹ and Z² groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹ and Z² groups is not a hydrogen atom, (4) R is the same or different carbyl groups having between 1 and 3 carbon atoms, (5) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (6) the R^{a2-a4} may be linear or branched carbyl groups having between about 12 to 40 carbon atoms, (7) the R^{d,f,x} groups may be hydrogen atoms or linear or branched carbyl groups having between 1 and 10 carbon atoms, and (8) R^{g,h,i,w,z} are linear or branched carbyl groups having between 1 and 10 carbon atoms. In certain embodiments, at least two of the Z¹, Z² and Z³ groups are not a hydrogen atom. In other embodiments, all of the Z¹, Z², and Z³ groups are not a hydrogen atom.

Embodiments of this invention systematic studies self-assembled monolayers (SAMs) derived from a series of four different new adsorbates designated Series I, Series II, Series III, and Series IV, as described in the subsequent paragraphs. Such systematic study helps establish the fundamental guidelines for the molecular designs and structure-property relationships of the organic monolayer films based on these aromatic chelating adsorbates. Of particular interest here are investigations that focus on the differences in the commensurability between the chelating head groups and the numbers of hydrocarbon chain tail groups, structural conformations, molecular packing densities, chain packing densities, and thermal stabilities for these monolayers. Such investigations lead to a better understanding of the factors that govern structurally complex monolayer interfaces and allow us to generate and study model interfaces that mimic the complexity found in the nature (e.g., cell surface) and in advanced applied materials.

### Series I (R1ArMT, R1ArDT, and R1ArTT)

This series includes adsorbates of general formula R1ArMT, R1ArDT, and R1ArTT, where R1=alkyl or alkoxy chain (e.g., —CₙH₂ₙ₊₁ or —OCₙH₂ₙ₊₁), Ar=aromatic moiety (e.g., —C₆H₄), MT=monothiol (e.g., —CH₂CH₂SH), DT=dithiol (e.g., —CH(CH₂SH)₂), and TT=trithiol (e.g., —C(CH₂SH)₃). The aromatic-based adsorbate molecules in this series are comprised of tri, di, and monothiolate head groups connecting to the intervening benzene ring with the single chain of hydrocarbon tail group placed at the opposite position with respect to the head group, as shown below:

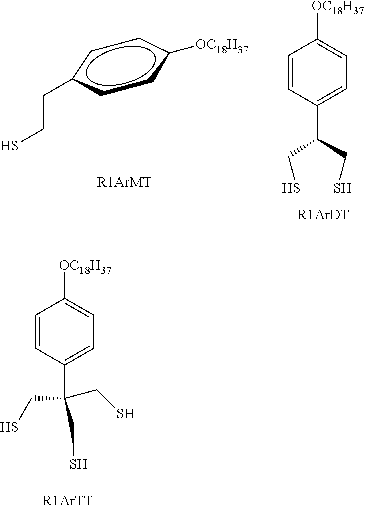

In this series, the present invention teaches the influence of the multidentate head groups on the structure-property relationships, especially, thermal and chemical stability.

### Series II (R²ArMT, R²ArDT, and R²ArTT)

This series includes adsorbates of general formula R²ArMT, R²ArDT, and R²ArTT, where R²=two alkyl or alkoxy chains (e.g., —CₙH₂ₙ₊₁ or —OCₙH₂ₙ₊₁) at the 3,5-positions of the aromatic ring, Ar=aromatic moiety (e.g., —C₆H₃), MT=monothiol (e.g., —CH₂CH₂SH), DT=dithiol (e.g., —CH(CH₂SH)₂), and TT=trithiol (e.g., —C(CH₂SH)₃). The molecular structures of these adsorbates are shown below:

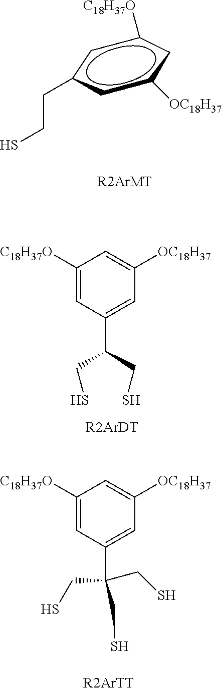

### Series III (R3ArMT, R3ArDT, and R3ArTT)

This series includes adsorbates of general formula R3ArMT, R3ArDT, and R3ArTT, where R3=three alkyl or alkoxy chains (e.g., —CₙH₂ₙ₊₁ or —OCₙH₂ₙ₊₁) at the 3,4,5-positions of the aromatic ring, Ar=aromatic moiety (e.g., —C₆H₃), MT=monothiol (e.g., —CH₂CH₂SH), DT=dithiol (e.g., —CH(CH₂SH)₂), and TT=trithiol (e.g., —C(CH₂SH)₃). The molecular structures of these adsorbates are shown below:

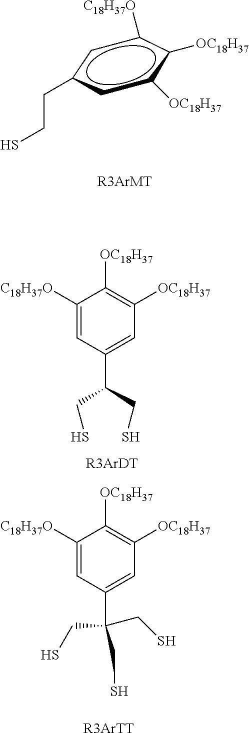

The rationale for such designs in Series II and III is that an increased number of the tail groups can increase chain assembled interactions for SAMs derived from bi- and tridentate adsorbates, which should enhance the chain packing, which in turn should enhance film stability.

### Series IV (R1ArmDT, R2ArmDT, R3ArmDT, and R2CpDT)

This series includes adsorbates of general formula R1ArmDT, R2ArmDT, R3ArmDT, and R2CpDT, where R1, R2, and R3=one, two, and three alkyl or alkoxy chains (e.g., —CₙH₂ₙ₊₁, or —OCₙH₂ₙ₊₁) at the 5-, 4,6-, and 4,5,6-positions of the aromatic ring, respectively, Ar=aromatic moiety (e.g., —C₆H₃, —C₆H₂, —C₆H₁, and pyridine analogs), Cp=five-membered ring analogs (e.g., cyclopentadiene, pyrrole, furan, or thiophene), and mDT=meta-dithiol (e.g., —CH(CH₂SH)₂). In this series, the present invention teaches the influence of the intramolecular S—S distances on the structure-property relationships, particularly the film stability. The meta-dithiol architecture for these adsorbates with tail groups incorporated at the 5-, 4,6-, and 4,5,6-position on the aromatic ring is shown below:

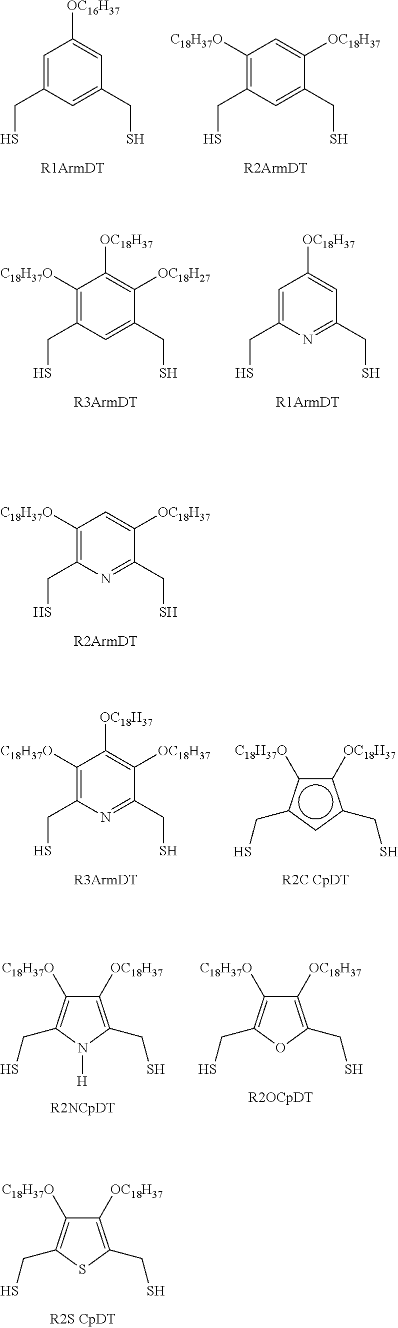

The rationale for this design is that the thiol groups are positioned in a manner that inhibits or excludes the formation of intramolecular cyclic disulfides, which should lead to films with enhanced stability by inhibiting or eliminating a key desorption pathway.^{20,21} A comparison of the characteristics and thermal stabilities of films derived from R1ArDT and R1ArmDT supports this hypothesis.

The evaluation of the quality of monolayer films on metal substrates is determined by measuring the thicknesses of the resultant films using ellipsometry.²² To understand the relationship between film thicknesses and molecular orientations as well as conformational orders of the SAMs, it is useful to compare the ellipsometric thicknesses of the monolayers with the theoretical thicknesses estimated from the models,²³ as shown in Table 1.

TABLE 1
Calculated Thicknesses and Measured Thicknesses for SAMs Generated
from C18 and New Classes of Multidentate Aromatic Thiols

| Adsorbate | Calculated Thickness (Å) | Measured Thickness (Å)* |
|---|---|---|
| C18 | 22 | 22 |
| R1ArMT | 28 | 28 |
| R1ArDT | 31 | 19 |
| R1ArmDT | 29 | 18 |
| R1ArTT | 31 | 18 |
| R2ArMT | 28 | 29 |
| R2ArDT | 30 | 23 |
| R3ArMT | 28 | 29 |
| R3ArDT | 30-31 | 29 |

*The ellipsometric thicknesses were reproducible within ±2 Å

Embodiments of the present invention disclose that the ellipsometric thickness of SAMs derived from single-tailed adsorbates decreases with an increase of the numbers of chelating head groups, going from mono, di, and tridentate thiols. This is clearly observed in Series I (with adsorbates R1ArMT, R1ArDT, and R1ArTT). These results are consistent with a change in the sulfur-to-chain ratios of the adsorbates, which is 1:1 for R1ArMT, 2:1 for R1ArDT and 3:1 for R1ArTT, namely, a size mismatch between head groups and the single chain tail group. The creation of void space between individual molecules due to the incommensurability between the relatively larger size of the multidentate head groups and the single hydrocarbon chained tail group results in the weak intermolecular interactions,²⁴ for example, π-π stacking interactions and inter-chain interactions. Consequently, these cause the deformation and tilt of the molecular chains. With the comparison of the ellipsometric thicknesses to the theoretical values, we therefore quantitatively conclude that the degree of chain tilt of these monolayers increases as following: R1ArTT>R1ArDT>R1ArMT, while the trends in the chain conformational order exhibits the reverse trend from the relative chain tilt.

Other embodiments of the present invention disclose that the ellipsometric thickness of SAMs increase with increasing the numbers of branched chains, namely chain-to-sulfur ratios. This is observed in the comparisons of monolayers derived from multi-tailed adsorbates (R3ArMT, R2ArMT, R3ArDT, and R2ArDT) with those derived from single-tailed adsorbates (R1ArMT and R1ArDT). For example, the thickness values of SAMs generated from mono and bidentate adsorbates having one, two, and three tail groups increases as follows: R3ArMT, R2ArMT>R1ArMT, and R3ArDT>R2ArDT>R1ArDT, respectively. The increase of the chain-to-sulfur ratios diminishes the void volume above the phenyl ring, which therefore increases the chain packing density and the chain-chain interactions. Comparison of the estimated thicknesses with the measured thicknesses (see Table 1) reveals good agreement for the SAM derived from R1ArMT, but a slight underestimation for the SAMs derived from R2ArMT and R3ArMT. These results can be interpreted to indicate that the molecules of the R1ArMT SAMs tilt approximately 30° from the surface normal with the chains possessing a largely trans zig-zag conformation. On the other hand, the underestimated thicknesses for the R2ArMT and R3ArMT SAMs can be interpreted to indicate that the molecules actually tilt less than 30° and/or that the molecules pack more densely on the surface those in normal SAMs. In addition, the significantly lower measured thickness values of R1ArDT and R2ArDT SAMs as compared to those obtained from the molecular models can be rationalized as a consequence of the high degree of chain tilt and chain deformation due to the less chain assembled interactions.²⁴ In contrast, the ellipsometric thickness of the R3ArDT SAM is close to that obtained from the model, which suggests that the long alkoxy chains in R3ArDT monolayer are likely tilted in less degree with respects to the surface normal as compared to that in R2ArDT and R3ArDT SAMs. As whole, the results reflect that the chain-assembled interactions in the R3ArDT SAM is higher than those in the R1ArDT and R2ArDT SAMs. Based on the ellipsometric thickness data, the relative chain tilt of the SAMs generated from these new adsorbates can be qualitatively estimated as follows: R1ArDT>R2ArDT>R3ArDT, while the trends in the chain conformational order exhibits the reverse trend from the relative chain tilt.

Other embodiments of the present invention disclose that the slight variation in the S . . . S distance for the different designs of dithiolate head groups does not significantly influence film thicknesses. This is observed in the comparison of monolayers derived from R1ArDT and R1ArmDT. The molecular lengths determined from the models of R1ArDT and R1ArmDT exhibit in the same extent with 2 Å difference. Therefore, the ellipsometric thickness values for both SAMs could be qualitatively compared to discern their structural formation (i.e., chain tilt and molecular packing density). Considering the two different designs of the dithiolate head groups for these two adsorbates, R1ArmDT has a longer intramolecular S . . . S distance than R1ArDT. Accordingly, R1ArmDT would occupy a larger surface area and create a larger space between the alkyl chains above the aromatic ring than R1ArDT. To optimize the inter-chain interactions, the long hydrocarbon chains in the R1ArmDT monolayer would be more tilted than those in the R1ArDT monolayer. The ellipsometric thickness of the R1ArmDT monolayer would therefore display a noticeably lower value when compared to that of the R1ArDT monolayer. In fact, both monolayers possess the same order of the ellipsometric thickness with only 1 Å difference (19 Å and 18 Å for R1ArDT and R1ArmDT monolayers, respectively). The result therefore implied a similarity in the average chain tilt for both monolayers. In addition, this data may also suggest that R1ArmDT might occupy at the binding sites resemble and/or closely to those of R1ArDT, in which the inter-chain distances associated with chain-chain interactions for both monolayers would be in the same extent. The theoretical calculation demonstrates that the adsorption of both sulfur atoms of the 1,3-benzenedimethanethiol (1,3-BDMT) locates at the bridge site on the gold surface. Furthermore, it was reported that the distance between sulfur atoms for dithiolate head group in R1ArDT can span as far as 4.8 Å without introducing bond-angle strain,²⁵ which is in the same extent of the intramolecular spacing between sulfur atoms in R1ArmDT (˜4.43 Å).

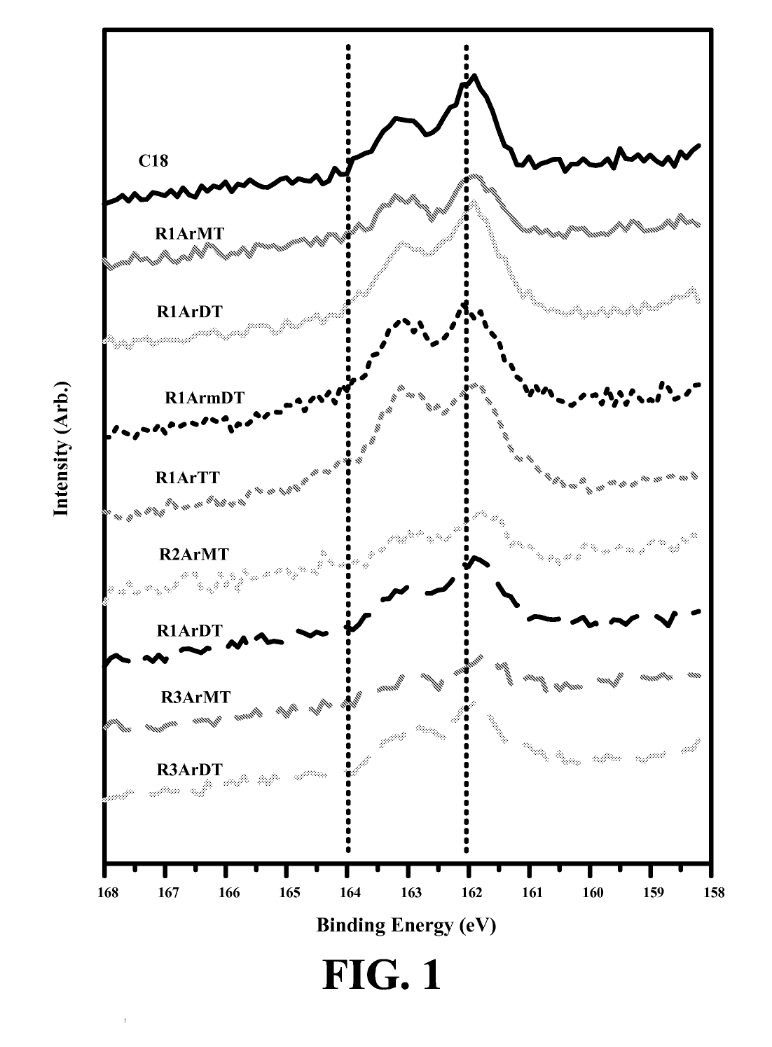

Other embodiments of the present invention teach that SAMs generated from the mono, and bidentate aromatic thiols are covalently bound to sulfur atoms on the gold surface. The XPS analysis for the peak positions of the S 2p region (shown in FIG. 1) reveals that the SAMs generated from the mono, and bidentate aromatic thiols possess a binding energy of ca. 162-163.2 eV, indicating the presence of fully bound sulfur atoms on gold.²⁶ On the other hand, the SAM generated from the tritentate aromatic thiol, R1ArTT, displays the shoulder peak at ˜164-165 eV, suggesting the incomplete binding of sulfur atoms on gold.^{26,27} The deconvolution of S 2p spectrum of the R1ArTT SAM revealed the ˜85% of all sulfur atoms bound to the gold surface. Furthermore, no oxidized sulfur species (i.e., sulfoxide, and sulfone) at binding energy ˜166-168 Ev²⁸ were detected for all monolayers, indicating the integrity of the monolayer formations.

Further XPS analysis on the peak integration ratios of S/Au²⁹ (data not shown) provides the information of molecular packing densities of the monolayers.^{18,30,31} For example, in the present invention, the relative molecular packing densities of the monolayers are compared to the densely packed C18 SAM as a reference, as shown in Table 2. (We noted that, for the adsorbates with a single chained tail group, the molecular packing density therefore reflect the chain packing density).

TABLE 2
Relative Molecular Densities, Relative Chain Packing Densities, and
Possible Maximum Tilt Angles of SAMs Derived from C18 and New
Classes of the Multidentate Aromatic Thiols Calculated from XPS Data

| | | | Possible |
|---|---|---|---|
| | Relative Molecular | Relative Chain | Maximum Tilt |
| Adsorbate | Packing Density* | Packing Density | Angle (degree) |
| C18 | 100 | 100 | 30 |
| R1ArMT | 81 | 81 | 36 |
| R1ArDT | 63 | 63 | 43 |
| R1ArmDT | 55 | 55 | 47 |
| R1ArTT | 58 | 58 | 46 |
| R2ArMT | 68 | 136 | 23 |
| R2ArDT | 49 | 98 | 31 |
| R3ArMT | 44 | 132 | 24 |
| R3ArDT | 44 | 132 | 24 |

*We estimate the experimental error in the packing density to be ±4%.

Other embodiments of the present invention disclose that the relative molecular packing densities of the SAMs derived from single-tailed adsorbates decrease proportionally with a decrease of the chain-to-sulfur ratios. This is observed in Series I where the relative molecular packing densities of the SAMs decrease proportionally with an increase of the coordinated sulfur atoms. The trend of molecular packing density increases as follows: R1ArMT>R1ArDT>R1ArTT.

Other embodiments of the present invention teach that the increasing numbers of the hydrocarbon chains decreases molecular packing densities of the SAMs. This is observed in the comparisons of monolayers derived from the multi-tailed adsorbates (R3ArMT, R2ArMT, R3ArDT, and R2ArDT) with those derived from single-tailed adsorbates (R1ArMT and R1ArDT). For example, the molecular packing densities of SAMs generated from mono and bidentate adsorbates having one, two, and three tail groups increase as follows: R1ArMT>R2ArMT>R3ArMT and R1ArDT>R2ArDT>R3ArDT, respectively. On the other hand, the chain packing densities increase as follow: R2ArMT˜R3ArMT>R1ArMT and R3ArDT>R2ArDT>R1ArDT, which show the corresponding values of 136%, 132%, and 82% for the former trend and 63%, 98%, and 132% for the later trend, respectively (shown in Table 2), due to the ratios of alkoxy chains to head groups for these adsorbates.

Accordingly, the trends of molecular packing densities for the monolayers derived from these mono and bidentate adsorbates can be clearly explained by the basis of the incommensurability between the thiolate head groups and the long hydrocarbon chain tail groups. The greater mismatch of the molecular counterparts requires the larger space for occupying the molecules, resulting in lower molecular packing densities.

Other embodiments of the present invention teach that the molecular packing density slightly decreases with longer intramolecular S . . . S distance. This is observed in the comparison of SAMs derived from R1ArDT and R1ArmDT, where the value of the molecular packing density of the latter monolayer is slightly lower when compared to that of the former monolayer. That can be partially attributed to the longer intramolecular S . . . S distance of R1ArmDT, which occupies a larger surface area than R1ArDT. Moreover, the results thus far imply that the orientation of the benzene ring in the R1ArmDT monolayer would be in an upright geometry; however, it would be more slightly tilted towards the metal surface than that in the R1ArDT monolayer. This can be attributed to the shorter distance between the benzene ring and the surface in the R1ArmDT film, leading to the partial contribution of the p-metal interaction on the formation of the monolayer which may induce and cause the tilt of the ring.^{32,33}

Other embodiments of the present invention teach to analyze the orientation of the alkyl chains by comparing the relative chain packing densities of these monolayers. This is done by roughly estimating the possibility of the maximum chain tilts of these SAMs based on the molecular packing densities by using a densely packed C18 monolayer with the chain tilt of 30 and by assuming that alkyls chains for SAMs in this study are fully-trans extended conformation. The estimated maximum chain tilts of SAMs derived from the new classes of multidentate aromatic thiols are shown in Table 2.

The possible maximum chain tilt of the R1ArMT, R1ArDT, and R1ArTT SAMs are approximately 36°, 43°, and 46°, respectively (shown in Table 2). As mentioned above, the estimated chain tilts for the monolayers in this series point out that the tilt angles increase with increasing the molecular incommensurability between head group and the single chained tail group, namely sulfur-to-chain ratios. The occupying larger sizes of the multidentate head groups on the underlying gold surface creates void space between the alkyl chained tail groups. Thus, the tilts of alkyl chains increase in order to optimize inter-chain interactions. This interpretation is consistent with the chain tilt inferred from ellipsometric data and the results from PM-IRRAS and wettabilty experiments, which will be discussed in subsequent sections.

Based on the measured packing density of the tail groups and assuming that the alkyl chains are fully trans-extended, we can estimate the maximum possible tilt of the alkyl chains of R1ArMT, R2ArMT, and R3ArMT SAMs to be 36°, 23°, and 24°, respectively (shown in Table 2). This analysis suggests that the average tilt of the chains in these SAMs is distinct for each of the adsorbates examined and further distinct from the 30° chain tilt of normal alkanethiolate SAMs on gold. Furthermore, the nature and magnitude of the tail group packing densities can influence both the assembly processes and the resultant structural quality of the films,^{22,24,34,35} which will be discussed in the following sections.

Considering a densely chain packed R3ArDT monolayer (132%, relative to the C18 SAM), the data suggests that R3ArDT might afford SAM with the reduction of chain tilt and less space area per chain as compared to other adsorbates. In the case of R2ArDT, given the chain-to-head group ratio of 1:1, the same as C18, a slight difference in chain packing density for the R2ArDT SAM (98%, relative to the C18 SAM) implies that the alkyl chains in the R2ArDT SAM might be tilted approximately ˜30 from the surface normal. On the other hand, the loosely chain and molecular packing densities for the monolayers derived from R1ArDT, due to the its incommensurability between the aromatic dithiolate headgroup and the single chained tail groups, give rise to a significant increase in the chain tilt. The possible maximum chain tilt of the R1ArDT, R2ArDT, and R3ArDT SAMs are approximately 43°, 31°, and 24°, respectively. These interpretations are consistence with the previous studies, which have been reported that an increase of van der Waal diameters of tail group segments results in a reduced tilt angle.^{36,37}

Due to its slightly lower chain packing density as compared to that of the R1ArDT monolayer, the R1ArmDT monolayer is expected to possess alkyl chains with a similar degree of chain tilt in the R1ArDT monolayer. The possible maximum chain tilts of the R1ArDT and R1ArmDT SAMs are approximately 43° and 46°, respectively. The calculated chain tilted point out that the alkyl chains for the R1ArmDT monolayer is more likely to have a higher exposure of methylene unit to the surface than that for the R1ArDT monolayer. This interpretation is strongly supported by the wettability data, which are discussed in the subsequent section.

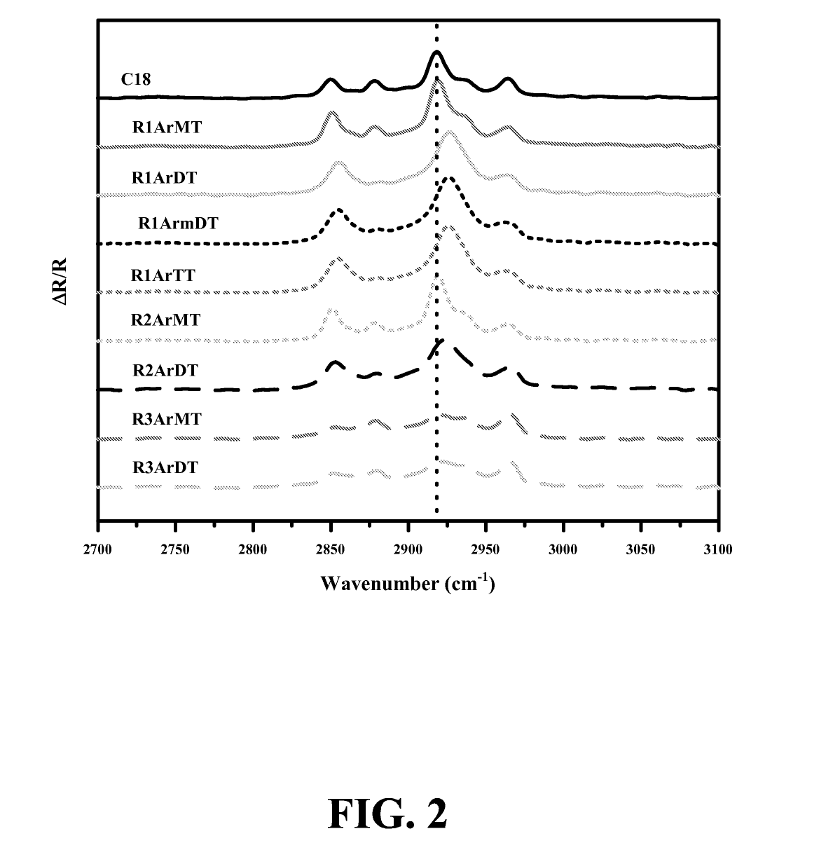

Other embodiments of the present invention teach methods for analyzing the conformational order and orientation of SAMs using PM-IRRAS. PM-IRRAS affords conformational order and orientation information regarding organic thin films.³⁸⁻⁴⁰ The frequency and band width of the methylene asymmetric C—H stretch (νₐ^{CH}₂) are particularly sensitive to the degree of conformational order (crystallinity) of the hydrocarbon chains.³⁸ It is known that the densely packed and crystalline-like monolayer derived from C18 displays the position of the νₐ^{CH}₂ band at 2918 cm⁻¹. The shifts of this band to higher frequencies indicate less conformationally ordered monolayers.³⁸ PM-IRRAS spectra in C—H starching region of SAMs derived from the adsorbates in this study are shown in FIG. 2.

The PM-IRRAS spectra for the monolayer films in Series I revealed that the νₐ^{CH}₂ band for the R1ArMT SAM is located at the same position of 2918 cm⁻¹ when compared to that for the C18 monolayer, indicating a well-ordered structure of the R1ArMT SAM. On the other hand, the R1ArDT and R1ArTT SAMs possess broader bands and shift drastically to higher frequency at −2926 and 2927 cm⁻¹, respectively. Furthermore, the orientation (i.e., chain tilt) of the hydrocarbon chains can be qualitatively determined by the PM-IRRAS spectra. According to the surface selection rule, only the transition dipole moments that polarized and are perpendicular to the surface can be detected and observed in the vibrational spectrum.^{38,41} In the case of monolayers derived from n-alkanethiols on gold with trans-extended alkyl chains containing an even number of carbon atoms, the dipole moment of the symmetric methyl stretching vibration (νₛ^{CH}₃) is nearly perpendicular to the surface,^{42,43} resulting in a significantly stronger intensity for the corresponding peak. With an increasing chain tilt from the surface normal, the intensity of the νₛ^{CH}₃ will decrease and the chains will exhibit a greater exposure of methylene units to the surface.^{2,43} The PM-IRRAS spectra in FIG. 2 demonstrate a substantial decrease in the intensities of the νₛ^{CH}₃ bands in the monolayers derived from R1ArDT and R1ArTT when compared to those derived from C18 and R1ArMT. Qualitatively, the degree of chain tilt for all these monolayers with respect to the νₛ^{CH}₃ intensities increases in the following order: C18˜R1ArMT>R1ArDT>R1ArTT. Additionally, the broader and enhanced intensities of νₐ^{CH}₂ vibrational mode also support the conclusion that the presence of higher chain tilts and conformational disordering of the hydrocarbon chains in R1ArDT and R1ArTT monolayers is impacting the data, where a great number of νₐ^{CH}₂ transition dipole moments are oriented along the surface normal due to the more random orientation of the methylene units.^{41,44,45} However, it should be noted that PM-IRRAS suppresses signal from randomly oriented film components, leading to absolute intensities that can vary from sample to sample.^{46,47} As a whole, the interpretations from the PM-IRRAS spectra are in good agreement with the results from the ellipsometric thickness measurements, and the packing densities of the monolayers investigated by XPS spectra and contact angle measurements.

For SAMs derived from R1ArMT, R2ArMT, and R3ArMT, the νₐₛ^{CH}₂ and νₛ^{CH}₂ bands for the SAM derived from R1ArMT appear at 2918 cm⁻¹ and 2851 cm⁻¹, the same as that found for C18, indicating a crystalline-like conformational order for both films. The νₐₛ^{CH}₂ bands of R2ArMT and R3ArMT films broaden and shift to higher frequency at 2923 and 2920 cm⁻¹, respectively. These results indicate that the SAMs derived from the double- and triple-chained adsorbates are less conformationally ordered than those derived from C18 and R1ArMT. Overall, the IR data suggest the following order for the relative order/crystallinity of the SAMs: C18˜R1ArMT>R3ArMT>>R2ArMT. The data for the SAM derived from R2ArMT are particularly striking and indicate a liquid-like conformation for the alkyl chains. To discern the interplay of chain packing and orientation induced by the branched chains, further interpretation of the IR spectra is required. The νₛ^{CH}₃ bands of the SAMs generated from R2ArMT and R3ArMT are more intense than those generated from C18 and R1ArMT, which can be interpreted to indicate that the terminal methyl groups in the R2ArMT and R3ArMT SAMs are oriented more closely to the surface normal than those in the C18 and R1ArMT SAMs. The broad region from 2890-2950 cm⁻¹ arises from three overlapping components: the anti-symmetric methylene stretching band (νₐₛ^{CH}₂˜2918-2924 cm⁻¹) and the Fermi resonances (FR) of the symmetric methylene stretching band (νₛ^{CH}₂ FR ˜2890) and the symmetric methyl stretching band (νₛ^{CH}₃ FR ˜2940).^{39,40,48,49} The Fermi resonance bands provide useful information regarding intermolecular interactions and chain assemblies. In the Raman spectra of n-alkanes, for example, Snyder et al. reported that the half-width of the νₛ^{CH}₃ FR band is broader in the neat crystal than that in the isolated matrix,^{49,50} which suggests that the bandwidth increases with increasing chain-chain interactions and chain packing.^{39,40,48} Furthermore, the same phenomenon was observed for the νₛ^{CH}₂ FR band in IR spectra, but the magnitude of the broadening was diminished because of a large difference in frequency between the fundamental and the binary state.⁴⁹ Qualitatively, the relative broadness of the Fermi resonances in FIG. 2 suggest that chain packing densities are greater in the SAMs derived from R2ArMT and R3ArMT than in the SAMs derived from C18 and R1ArMT, which is consistent with our interpretation of the XPS data (vide supra). Another interesting observation is the decrease in the intensity of the bands related to methylene stretching in the R2ArMT and R3ArMT SAMs compared to the corresponding bands in the C18 and R1ArMT SAMs. On the basis of the surface selection rules for IR spectroscopy, the intensities of both methylene stretching modes decrease with decreasing chain tilt.^{45,51,52} Thus, the diminished intensity of the methylene stretching bands might indicate a smaller average tilt angle for the R2ArMT and R3ArMT SAMs than that in the C18 and R1ArMT SAMs, assuming that all of the monolayers in are isotropic and composed of all trans-extended chains. Moreover, a reduced chain tilt (<30°) for the SAMs derived from R2ArMT and R3ArMT is in good agreement with the ellipsometric thickness data in Table 1, particularly when one considers the almost certainly poor molecular packing underneath the bulky aromatic units of R2ArMT and R3ArMT. However, despite the trends noted in the IR data here, we caution that changes in frequency, intensity, and bandwidth can arise from a variety of other factors, including chain deformations^{53,54} and differences in the twist angles along the axis of the long alkyl chains.⁵⁵⁻⁵⁷

For SAMs derived from the new bidentate aromatic thiols, R1ArDT, R2ArDT, and R3ArDT, the positions of the νₐₛ^{CH}₂ bands shift to higher wave numbers ˜2925, 2923, and 2920 cm⁻¹, respectively. The two former SAMs represent the substantial shifts of the νₐₛ^{CH}₂ bands, indicating that they are less crystalline in nature when compared to the later SAM where the νₐₛ^{CH}₂ band shifted slightly higher (˜2 cm⁻¹) relative to that of the crystalline-like conformational structure of the C18 SAM. The lower crystallinity of the R3ArDT monolayer compared to the C18 monolayer can be attributed to the structurally constrained hydrocarbon chains in the R3ArDT SAM, which might lead to partially disordered alignment of the chains. Therefore, based on the position of νₐₛ^{CH}₂ band, the degree of conformation order of these monolayers decreases in the following order: C18>R3ArDT>R2ArDT>R1ArDT. Another interesting feature of the IR spectra in the C—H stretching region in FIG. 2 is the presence of the broad bands of the Fermi resonance of the symmetric methyl (νₛ^{CH}₃ FR) appearing at ˜2935 cm⁻¹ in the spectra of the C18 and R3ArDT monolayers. On the other hand, in the case of the R1ArDT and R2ArDT films, the contribution of the Fermi resonance bands are less significant and fully overlap with the νₐₛ^{CH}₂ bands. It has been reported that the predominant Fermi resonance band is indicative of a strong chain-chain interactions.^{39,40,48-50} Therefore, the results point out that the strong influence of the chain assembly interactions apparently exerts a greater impact on the monolayer films derived from the C18 and R3ArDT monolayers, while presenting less influence on the R1ArDT and R2ArDT monolayers. Taking all IR results together, we discovered that the conformational order and the interplay of chain-chain interactions for the monolayers derived from these chelating adsorbates increase as the numbers of branched chains increase. This can be rationalized in terms of the chain packing density, namely chain-to-head group ratios, and commensurability between the dithiolate head group and branched chain tail groups. For example, in the case of the R3ArDT monolayer, the cross-sectional area of the triple-chained hydrocarbon branches is larger than the area occupied by the dithiolate head group on the gold surface, causing a loosely molecularly-packed monolayer and exhibiting less inter-chain interactions between two neighboring adsorbate chains. However, the presence of the well-ordered conformation of the hydrocarbon chains is due to the compensation of the intramolecular chain-to-chain interaction themselves among three long alkoxy chains, which is strongly supported by the PM-IRRAS and XPS data inferred by the νₛ^{CH}₂ FR and a densely chain packing (132%), respectively. On the other hand, for the R1ArDT monolayer, the aromatic dithiolate head group is a mismatch to the single chain tail group, in which the cross-section area of the hydrocarbon chain is smaller relative to the area occupied by the head group. Although the molecular packing density of the R1ArDT monolayer is more densely relative to that of the R2ArDT and R3ArDT monolayers, the incommensurability between the head group and the single chain hydrocarbon in R1ArDT creates a larger void space above the phenyl ring. This causes a loosely chain packing with a higher chain tilt and deformation of the long alkoxy chains due to a reduction in chain assembly (e.g., inter- and intramolecular chain-to-chain interactions). Given the ratio of the sulfur head group to the long alkoxy chains of 1:1, as is the case with C18, R2ArDT represents the chain packing density of 98%, which slightly deviate from the densely packed C18 monolayer. Indeed, the addition of two long alkoxy chains improves the conformational order of the R2ArDT SAM when compared to that of R1ArDT SAM; however, it still presents a less ordered film than that of the C18 SAM. These results can be attributed to the large dimension of the aromatic chelating head group and also to the void space between the double-branched chains that perturb the molecular packing density and diminish the chain-chain interactions of the R2ArDT SAM.

Compared with the peak characteristic of the νₐₛ^{CH}₂ for the C18 SAM, the broader bandwidth and higher intensities of the νₐₛ^{CH}₂ bands with their position at higher frequencies ˜2926 cm⁻¹ for the R1ArDT and R1ArmDT SAMs indicated the deformation and disorder of the hydrocarbon chains (gauche conformation). Additionally, the barely visible peaks of the symmetric stretching mode of methyl (νₛ^{CH}₃) bands also evidenced the gauche conformation of the hydrocarbon chains for these two monolayers. As judged by the IR characteristic spectra, the degree of conformational order and orientation of hydrocarbon chains for the R1ArmDT and the R1ArDT monolayers is indistinguishable. However, by taking all aforementioned results from chain and/or molecular packing density and contact angle measurements of these two monolayer systems, it is reasonable to suggest that the tilts of the hydrocarbon chain and/or the aromatic ring in the R1ArmDT monolayer is slightly higher than those in the R1ArDT monolayer.

Other embodiments of the present invention teach methods for obtaining informative data of conformational order and chain orientations of monolayer films using contact angle measurements.⁵⁸ The contact angles data is shown in Table 3.

TABLE 3
Advancing (θₐ) and Receding (θᵣ) Contact Angles and Hysteresis
(Δθ = θₐ − θᵣ) for Water (H₂O) for Hexadecane (HD), and
Decalin (DEC) on Monolayer Films Generated from C18, and
Multidentate Aromatic Adsorbates

| | Contact Angle (degree)* | | | | | | | | |
|---|---|---|---|---|---|---|---|---|---|
| | Water | | | Hexadecane | | | Decalin | | |
| Adsorbate | θₐ | θᵣ | Δθ | θₐ | θᵣ | Δθ | θₐ | θᵣ | Δθ |
| C18 | 115 | 105 | 10 | 50 | 40 | 10 | 54 | 48 | 6 |
| R1ArMT | 115 | 105 | 10 | 50 | 40 | 10 | 54 | 48 | 6 |
| R1ArDT | 109 | 98 | 11 | 45 | 35 | 10 | 32 | 27 | 5 |
| R1ArmDT | 109 | 98 | 11 | 42 | 32 | 10 | 31 | 27 | 4 |
| R1ArTT | 107 | 97 | 10 | 42 | 32 | 10 | 27 | 20 | 7 |
| R2ArMT | 113 | 103 | 10 | 36 | 30 | 6 | 41 | 34 | 7 |
| R2ArDT | 111 | 101 | 10 | 42 | 32 | 10 | 38 | 32 | 6 |
| R3ArMT | 114 | 104 | 10 | 46 | 39 | 7 | 49 | 42 | 7 |
| R3ArDT | 113 | 103 | 10 | 44 | 34 | 10 | 47 | 37 | 10 |

*The average contact angles of water, hexadecane, and decalin were reproducible within ± 2 Å

The results of the advancing contact angles of water (θₐ^{H2O}) in Table 3 show an indication of the hydrophobic interfaces of all monolayers under the investigations. However, the sensitivity of water to probe hydrophobic monolayers is less than that of hydrocarbon solvents. Hexadecane is known to be a powerful tool for exploring the nanoscale of structural differences at the interfaces of hydrophobic films,^{54,59} although it can partially intercalate through loosely packed monolayer.^{18,60-62} This phenomenon causes the misleading interpretation for quality of monolayers. Our recent studies on interfacial wetting of SAMs derived from chelating adsorbates demonstrated that decalin is more applicable to analyze hydrocarbon films, especially loosely packed monolayers, than hexadecane.⁶² This is due to its bulk steric molecular structure of decalin impeding the intercalation phenomena during the contact angle measurements.

For SAMs generated from multidentate adsorbates having a single chained tail group, the contact angle data in Table 3 demonstrates that the R1ArTT surface is more wettable by hexadecane and decalin than the R1ArDT surface, while the contact angle values of the R1ArMT SAM are apparently indistinguishable from the C18 SAM, which is higher than those of the R1ArDT and R1ArTT SAMs. According to the atomic contact model, the results pointed out that the R1ArTT SAM exhibits a greater number of methylene units exposed to the interface with a higher degree of chain tilt when compared to the R1ArDT and R1ArMT SAMs. This interpretation is strongly supported by the tilt angles inferred by the XPS data. On the other hand, the interface of the R1ArMT monolayer consists mainly of the terminal methyl groups and is similar to the densely packed and well-ordered monolayer derived from C18. Therefore, the conformational order of the monolayers in this series can be estimated as follow: C18˜R1ArMT>R1ArDT>R1ArTT.

In the case of SAMs generated from monodentate adsorbates having the varying numbers of alkoxy chained tail groups, Table 3 shows that the contact angles for both hexadecane and decalin follow the same trend: C18˜R1ArMT>R3ArMT>>R2ArMT. The fact that contact angles of water, hexadecane, and decalin are the same for the SAMs derived from R1ArMT and C18 supports our proposal above that the packing and orientation of the R1ArMT SAM is similar to that of normal alkanethiolate SAMs. The reduced values for R3ArMT and particularly R2ArMT are somewhat surprising, but can be analyzed by considering both the packing density of the SAMs and the molecular structure of the adsorbates. Interestingly, the contact angle values of hexadecane (θₐ^{HD}) and decalin (θₐ^{DEC}) drop from their maximum values for the R3ArMT SAM and drop markedly lower for the R2ArMT SAM. This trend is inconsistent with the molecular packing densities measured by XPS, where the relative percent coverages are 100, 81, 68, and 44 for C18, R1ArMT, R2ArMT, and R3ArMT, respectively (vide supra). However, the corresponding tail group coverages are 100, 81, 136, and 132, respectively, due to the ratio of head group to tail groups for these adsorbates. Given that the contacting liquids probe the tail groups more than the head groups of these SAMs, we focus on the tail group packing density, orientation, and conformation to interpret the wettability data. As noted above regarding the XPS data, it is likely that the average tilt of the chains in the SAMs is distinct for each adsorbate. Despite this complication, it is still possible to infer structural/conformational information regarding the tail groups from the PM-IRRAS data. Specifically, the PM-IRRAS data indicate that the conformational order of the tail groups decreases as follows: C18˜R1ArMT>R3ArMT>>R2ArMT. Importantly, the advancing contact angles of hexadecane and decalin follow the exact same trend (see Table 3). This correlation is consistent with a model in which the tail groups in the SAMs derived from R3ArMT and especially R2ArMT are less conformationally ordered (i.e., possess more gauche conformations) and expose a higher fraction of methylene groups at the interface than the SAMs derived from C18 and R1ArMT. As detailed previously, interfacial methylene groups are more wettable than interfacial methyl groups.¹⁹

For SAMs generated from bidentate adsorbates having varying numbers of tail groups (R1ArDT, R2ArDT, and R3ArDT), Table 3 shows the unexpected and inconsistent results with no substantive difference in the interfacial wetting tested by hexadecane, in which θₐ^{HD} values fall within a range between about 42° and about 45°. We hypothesized that hexadecane may partially intercalate through the void space between adsorbate molecules, long alkyl chains and/or monolayer^{18,60,61} and interact with phenoxy adlayers, revealing θₐ^{HD} values which are found in the same extent for methoxy-terminated SAMs.⁶³ On the other hand, the contact angle values of decalin can be used to distinguish the interfacial difference of the SAMs derived from these dithol adsorbates. The investigation of decalin wettability for these monolayers exhibits the interesting tendency that the θₐ^{DEC} values decrease with a decrease of the number of branched chains. Specifically, the data suggests the ability of the branched chains to fill the void space above the phenyl rings and to form more trans-extended conformations through van der Waal interactions between chains. Therefore, the conformational order of the long alkoxy chains were interpreted to decrease according to the trend as follows: R3ArDT>R2ArDT>R1ArDT.

The comparison of the contact angle values of SAMs generated from the different designs of the dithiolate head groups shows that the θₐ^{HD} for the R1ArmDT monolayer is 3 less than the values for the R1ArDT SAM, while the difference in the θₐ^{DEC} between these two monolayers is less pronounced. This might be ascribed to the lesser atomic contact of decalin when compared to that of hexadecane. As a whole, the lower values of contact angles for both testing liquids for the R1ArmDT SAM as compared to those for the R1ArDT SAM indicate that the former monolayer has a greater numbers of methylene units exposed at the interface than the later monolayer as a result of the slightly higher tilts of the aromatic ring and/or the long chain hydrocarbon (as discussed in the previous section).

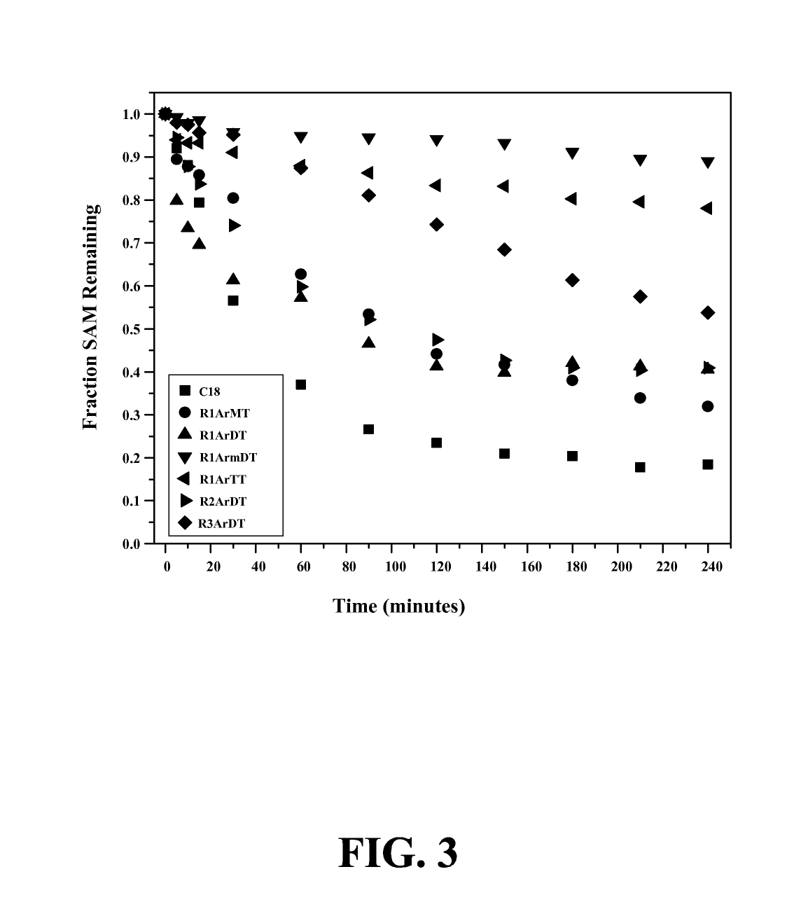

Other embodiments of the present invention teach methods for evaluating the thermal stability of SAMs as a function of the numbers of branched chains and chelating head groups they contain. Stabilities of SAMs derived from selected adsorbates (R1ArMT, R1ArDT, R1ArTT, R2ArDT, R3ArDT, and R1ArmDT) are investigated by monitoring the solution-phase desorption in isooctane at 80° C. as a function of time. The extent of desorption is monitored by tracking the change in ellipsometric thicknesses. The desorption profiles illustrate the fraction of SAM remaining on the surface upon thermal treatment, as shown in FIG. 3. All of the desorption profiles of the SAMs derived from the adsorbates in the present study except R1ArmDT clearly exhibit two distinct desorption regimes: (1) a fast initial desorption regimes described as the relatively steep slope as the period of time ˜O-90 minutes; and (2) a slower/nondesorbing regime described as the gentle slope as the period of time ˜90-240 minutes.

To quantitatively determine the relative rates of desorption in the fast desorbing regime, we evaluate the rate constant (k) for the desorption at 80° C. by fitting the data with the first order-kinetics according to equation 1:[54]

(Tₜ−T_{∞})/(T₀−T_{∞})=e^{-kt} (1)

where Tₜ is the thickness of the monolayer at time t, and T_{∞} is the thickness of the monolayer at the infinite time (˜30 hours). Table 4 shows the relative rate constants for the fast desorbing regimes and the fractions of SAM remaining on the surfaces for both distinct desorption regimes at the specific time at 90 and 240 minutes for the fast initial and the slow/non-desorbing regimes, respectively.

TABLE 4
Relative Rate Constants (k) for the Fast Desorbing Regime and
the Fractions of SAM Remaining On the Surfaces for
Both Desorption Regimes at the Specific Time of 90 and
240 Minutes for the Fast Initial and Slow/Non-Desorbing Regimes

| | Fast Regime (0-90 minutes) | | Slow Regime |
|---|---|---|---|
| | | Rate Constants | (90-240 minutes) |
| Adsorbate | Remaining | k × 10⁻² (min⁻¹) | % SAM remaining |
| C18 | 27% | 2.37 | 18% |
| R1ArMT | 53% | 0.99 | 32% |
| R1ArDT | 46% | 1.82 | 40% |
| R1ArmDT | 94% | (a) | 89% |
| R1ArTT | 86% | 0.10 | 78% |
| R2ArDT | 52% | 1.46 | 41% |
| R3ArDT | 81% | 0.42 | 54% |

(a) The rate constant cannot be evaluated due to the insignificant change of the % SAM remaining.

In the case of slow/nondesorbing regime, desorption data cannot be fitted to obtain quantitative kinetic data because of the statistically insignificant changes in ellipsometric thickness which is in the experimental error range. The qualitative analysis of the relative thermal stability can be evaluated by comparing the remaining fraction of monolayers at arbitrary time in this regime. Therefore, the desorption profiles in this regime reflects thermodynamic rather than kinetic phenomena.

The desorption profiles of the SAMs derived from multidentate adsorbates having a single tail group (Series I) apparently illustrated that the R1ArTT SAM retains a higher degree of thermal desorption than the R1ArMT, and R1ArDT SAMs in both desorption regimes. Additionally, the R1ArMT SAM represented the greater thermal resistance of desorption than the C18 SAM. This can be rationalized by the influence of additional π-π interactions between aromatic rings. Additionally, the steric repulsion of the aromatic ring can plausibly impede the intermolercular disulfide formation[21] for the R1ArMT SAM. Surprisingly, the rate constant of the desorption for the fast desorbing regime of the R1ArMT SAM is two times slower than that of the R1ArDT SAM, indicating that the R1ArMT SAM is more thermally stable than the R1ArDT SAM. Thus, the thermodynamic preference of the chelate effect and entropical disfavoring of desorption due to the intra- or intermolecular disulfide formations failed to correlate with the relative thermal stability of the R1ArDT monolayer upon the desorption process in the fast kinetic regime. This result can be rationalized on the basis of the structural features of the adsorbates. Prior molecular modeling of the spiro-dithiol head group demonstrated that the extended distance between two sulfur atoms can be spanned as far as 4.8 Å without introducing excessive bond-angle strain.²⁵ Therefore, the S . . . S spacing of the spiro-dithiol head group on the gold surface is not commensurable to occupy the 3-fold hollow sites,²⁵ while our previous work proposed that the sulfur atoms of the monothiol adsorbate, R1ArMT, may likely bind to the 3-fold hollow sites of the Au(111) with a spacing of 4.99 Å, similarly to the sulfur atoms of C18 bound onto the gold surface.^{64,65}

According to the two distinct binding site models mentioned above, the restriction of access to the binding sites for both dithiolate head groups (e.g., one sulfur atom on the 3-fold hollow and the other one on the top site of Au(111)) might contribute to a lower stability for the dithiol-based SAM. The influence of the restricted access to binding sites on thiolate desorption is supported by previous work from Walczak et al., who reported that alkanethiolate-based SAMs on gold substrates have been shown to desorb more readily from the terrace sites than from the step sites.⁶⁶ Additionally, the better relative crystallinity of the R1ArMT monolayer as compared to that of the R1ArDT monolayer should also influence desorption behavior. On the other hand, the most stable of the tridentate-based monolayer plausibly reveals that the entropy-driven chelating effect outweighs the bonding sites of sulfur atoms on gold lattice. In addition, the attachments of three sulfur atoms afford the R1ArTT SAM more thermodynamically stable than the R1ArDT SAM. For instance, considering the intramolecular desorption of the R1ArDT monolayer, the pathway would concurrently break two S—Au bonds to form disulfide formation. On the other hand, the intramolecular desorption of the R1ArTT monolayer is unfavorable, which can be explained by the fact that if two sulfur atoms would become unbound from the gold surface to form the cyclic disulfide product there is still one more sulfur atom bound to the surface. Furthermore, the cyclic disulfide formed by the desorption of two bound sulfur atoms from the gold surface can be readily reestablished to form on the surface.⁶⁷ Moreover, the intermoelucar desorptions of the R1ArTT monolayer are entropically disfavored more than those of the R1ArDT monolayer. For example, the R1ArTT monolayer requires the simultaneous breaking of six S—Au bonds, while in the case of the R1ArDT monolayer, only four S—Au bonds are needed for the formation of the dimer heterocycle. Therefore, the relative thermal stability of the monolayers in the fast kinetic regime can be concluded in the following trend: R1ArTT>R1ArMT>R1ArDT>C18.

For the slow/non desorbing kinetic regime, the remaining monolayer fractions of the SAMs derived from this series at 240 minutes decrease as follow: R1ArTT<R1ArDT<R1ArMT. Therefore, the relative long-term thermal stability of the monolayers can be concluded in the following trend: R1ArTT>R1ArMT>R1ArDT>C18, which correlated with the degree of chelation.

The desorption profiles (FIG. 3) and rate constants (Table 4) revealed that the thermal stability of the SAMs obtained from the bidentate adsorbates in this study increases with increasing chain conformation inferred by the PM-IRRAS data. This result therefore indicates the key role of intramolecular chain-to-chain interactions to stabilize monolayers, in which the strength of chain assembled interactions is proportional to the numbers of molecular chains, namely the chain packing density. Consequently, we can rationalize the correlation between the desorption behavior and the degree of chain conformation, in which the most ordered conformation (implying strong van der Waals interactions) of the long alkoxy chains affords the most resistance to thermal desorption.^{68,69} We therefore conclude that the thermal stability of these monolayers derived from the bidentate adsorbates increases as follow: R1ArDT>R2ArDT>R3ArDT.

The comparison of the thermal desorption profiles of the R1ArmDT and R1ArDT SAMs illustrates that the former SAM is more thermally stable as compared to the later SAM, in which the fraction of SAM remaining on the surface for the former SAM is significantly greater than that for the later SAM in both desorption regimes. Furthermore, the comparisons of the desorption profile of the R1ArmDT SAM with the other SAMs in this present study revealed that the monolayer derived from the specific design of extended dithiolate head group is even more thermally stable than those derived from tridentate aromatic thiol, R1ArTT, and other bidentate aromatic thiols (i.e. R2ArDT and R3ArDT). The greatest enhanced thermal stability of the R1ArmDT SAM is rationalized to the longer intramolecular S . . . S distance of the dithiolate head group of R1ArmDT which hinder the formation of the intramolecular cyclic disulfides upon the desorption process.

As a whole, the relative thermal stability of SAMs in this present study increases as follows: R1ArmDT>R1ArTT>R3ArDT>R2ArDT>R1ArDT>R1ArDT>R1ArMT>C18.

### EXPERIMENTS OF THE INVENTION

### Examples

### S1. Synthesis of the Intermediates used to Prepare Multidentate Aromatic Thiols with Varying Numbers of Hydrocarbon Chained Tail Group

The synthetic pathways used to prepare the intermediate compounds 1a and 2a for preparing the mono- and bidentate aromatic thiols (i.e., R1ArMT, R1ArDT, R2ArMT, and R2ArDT), as well as the tridentate aromatic thiol, R1ArTT, shown in Scheme Si. For the intermediate compounds, 3g, used to prepare R3ArMT, R3ArDT, and R3ArTT, the synthetic pathway is displayed in Scheme S2.

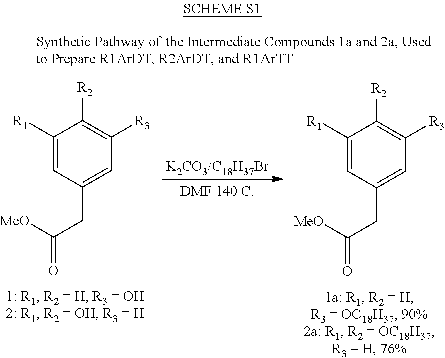

### 4-Octadecyloxy-phenylacetate (1a)

A mixture of K₂CO₃ (8.31 g, 60.2 mmol), methyl 4-hydroxy-phenylacetate (5.00 g, 30.0 mmol) and 1-bromoctadecane (13.04 g, 39.11 mmol) in DMF (80 mL) was stirred at 140° C. overnight. After cooling to rt, K₂CO₃ was removed by filtration, and the filtrate was diluted with H₂O and acidified with 2 M HCl. The aqueous layer was extracted with CH₂Cl₂ (3×250 mL). The organic layers were combined and washed with H₂O (3×50 mL), dried over MgSO₄ and concentrated to dryness to afford the crude product. The crude product was taken up in CH₂Cl₂ and then cold MeOH was added to precipitate 1a (11.30 g, 27.01 mmol, 90%) as white powder. ¹H NMR (500 MHZ, CDCl₃): δ 0.87 (t, J=6.9 Hz, ArOCH₂CH₂(CH₂)₁₅CH₃, 3H), 1.26-1.41 (m, ArOCH₂CH₂(CH₂)₁₅CH₃, 30H), 1.73 (pent, J=7.4 Hz, ArOCH₂CH₂(CH₂)₁₅CH₃, 2H), 3.56 (s, ArCH₂COOCH₃, 2H), 3.68 (ArCH₂COOCH₃, 3H), 3.93 (t, J=6.9 Hz, ArOCH₂CH₂(CH₂)₁₅CH₃, 2H), 6.85 (d, J=8.6 Hz, ArH, 2H), 7.17 (d, J=8.6 Hz, ArH, 2H).

### Methyl-2-(3,3-bis(octadecyloxy)phenyl)acetate (2a)

Following the procedures described for 1a, methyl-2-(3,5-dihydroxyphenyl)acetate (2.50 g, 13.8 mmol) was treated with K₂CO₃ (18.97 g, 137.2 mmol) and 1-bromooctadecane (13.73 g, 41.16 mmol) in DMF (150 mL) at 120° C. to give a pale yellow crude product. The crude product was purified by column chomatography using CH₂Cl₂:hexane (3:2) as the eluent to afford a white powder product 2a (7.21 g, 10.5 mmol, 76%). ¹H NMR (400 MHZ, CDCl₃): δ 0.87 (t, J=6.9 Hz, Ar(OCH₂CH₂(CH₂)₁₅CH₃)₂, 6H), 1.24-1.43 (m, Ar(OCH₂CH₂(CH₂)₁₅CH₃)₂, 60H), 1.74 (pent, J=7.8 Hz, Ar(OCH₂CH₂(CH₂)₁₅CH₃)₂, 4H), 3.53 (s, ArCH₂COOCH₃, 2H), 3.70 (s, ArCH₂COOCH₃, 3H), 3.90 (t, J=6.9 Hz, Ar(OCH₂CH₂(CH₂)₁₅CH₃)₂, 4H), 6.35 (s, ArH, 1H), 6.39 (s, ArH, 2H).

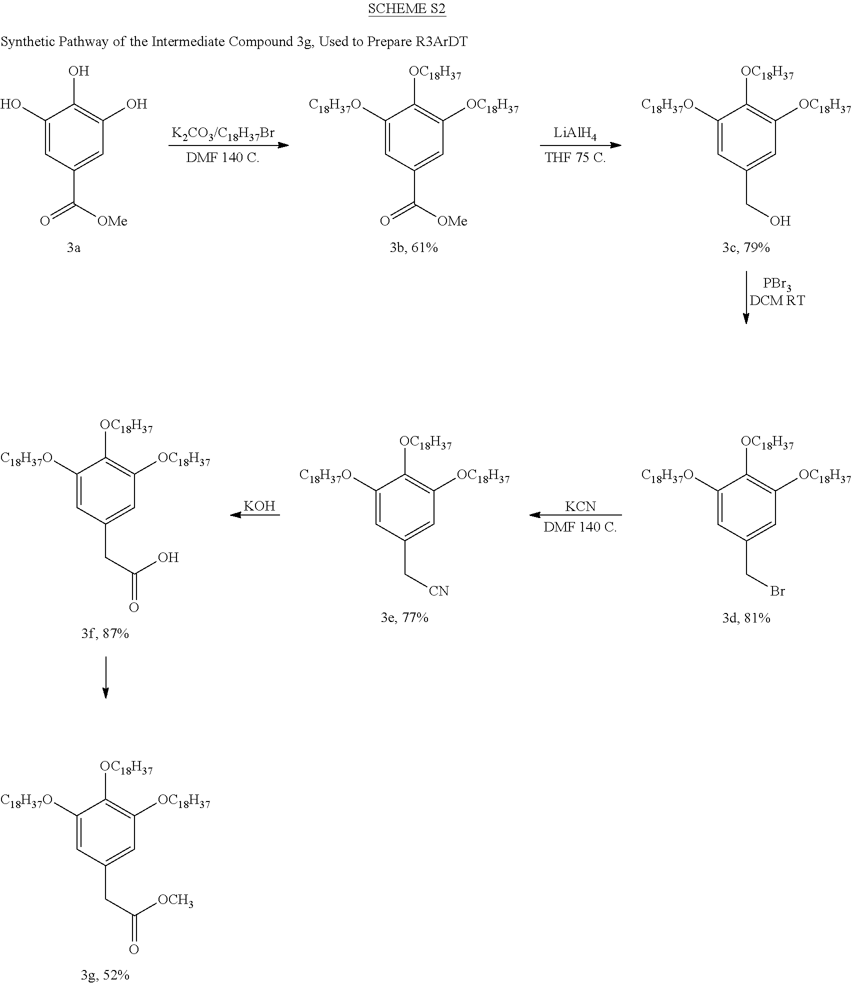

### Methyl-3,4,4-tris(octadecyloxy)benzoate (3b)

Following the procedure described for 1a, methyl-3,4,5-trihydroxybenzoate (2.00 g, 10.9 mmol) was treated with K₂CO₃ (22.53 g, 163.0 mmol) and 1-bromooctadecane (16.30 g, 48.90 mmol) in DMF (250 mL) at 140° C. to give a pale yellow crude product. The crude product was purified by column chomatography using EtOAc:Hexane (1:4) as the eluent to afford 3b as a white powder product (6.20 g, 6.58 mmol, 61%). ¹H NMR (500 MHZ, CDCl₃): δ 0.87 (t, J=7.2 Hz, Ar(OCH₂CH₂(CH₂)₁₅CH₃)₃, 9H), 1.24-1.48 (m, Ar(OCH₂CH₂(CH₂)₁₅CH₃)₃, 90H), 1.68-1.81 (m, Ar(OCH₂CH₂(CH₂)₁₅CH₃)₃, 6H), 3.87 (s, ArCOCH₃, 3H), 3.98-4.01 (m, Ar(OCH₂CH₂(CH₂)₁₅CH₃)₃, 6H), 7.24 (s, ArH, 2H).

### (3,4,5-Tris(octadecyloxy)phenyl)methanol (3c)

To a suspension of LiAlH₄ (0.60 g, 15 mmol) in THF (25 mL) was added dropwise a solution of 3b (6.00 g, 5.03 mmol) in THF (20 mL). The reaction mixture was refluxed for 6 h under argon, quenched with water, and acidified with 2 M HCl. After being stirred for 10 min, the resultant mixture was extracted with CH₂Cl₂ (3×150 mL). The combined organic layers were washed subsequently with brine (3×50 mL) and water (3×50 mL), dried over MgSO₄, and evaporated to dryness to give 3c (5.30 g, 5.80 mmol, 91%). ¹H NMR (500 MHZ, CDCl₃): δ 0.87 (t, J=7.2 Hz, Ar(OCH₂CH₂(CH₂)₁₅CH₃)₃, 9H), 1.24-1.47 (m, Ar(OCH₂CH₂(CH₂)₁₅CH₃)₃, 90H), 1.68-1.81 (m, Ar(OCH₂CH₂(CH₂)₁₅CH₃)₃, 6H), 3.63 (br, ArCH₂OH, 1H), 3.92-3.97 (m, Ar(OCH₂CH₂(CH₂)₁₅CH₃)₃, 6H), 4.59 (d, J=5.7, ArCH₂OH, 2H), 6.55 (s, ArH, 2H).

### 5-(Bromomethyl)-1,2,3-tris(octadecyloxy)benzene (3d)

A solution of PBr₃ (1.42 mL, 15.1 mmol) in CH₂Cl₂ (10 mL) was added slowly to a stirred solution of 3c (4.60 g, 5.30 mmol) in CH₂Cl₂ (100 mL) at rt. The mixture was continually stirred for 3 h under argon, quenched with H₂O (25 mL), and extracted with CH₂Cl₂ (3×100 mL). The combined organic layers were washed subsequently with brine (1×100 mL) and water (1×100 mL), and then dried over MgSO₄. Removal the solvents afforded 3d (4.00 g, 4.10 mmol, 81%) as a white powder product. ¹H NMR (500 MHZ, CDCl₃): δ 0.87 (t, J=6.9 Hz, Ar(OCH₂CH₂(CH₂)₁₅CH₃)₃, 9H), 1.24-1.47 (m, Ar(OCH₂CH₂(CH₂)₁₅CH₃)₃, 90H), 1.68-1.81 (m, Ar(OCH₂CH₂(CH₂)₁₅CH₃)₃, 6H), 3.92-3.96 (m, Ar(OCH₂CH₂(CH₂)₁₅CH₃)₃, 6H), 4.43 (s, ArCH₂Br, 2H), 6.56 (s, ArH, 2H).

### 2-(3,4,5-Tris(octadecyloxy)phenyl)acetonitrile (3e)

To a solution of 3d (4.20 g, 4.30 mmol) in DMF (150 mL) was added a solution of NaCN (1.05 g, 21.5 mmol) in water (15 mL). The mixture was heated at 140° C. for 72 h, quenched with water (50 mL), and extracted with CH₂Cl₂ (3×100 mL). The combined organic layers were washed subsequently with brine (1×100 mL) and water (1×100 mL), and dried over MgSO₄. The solvent was removed to dryness to obtain the crude product. The crude product was dissolved in a minimum volume of CH₂Cl₂, and then cold MeOH was added to precipitate 3e (3.13 g, 3.39 mmol, 79%). ¹H NMR (500 MHZ, CDCl₃): δ 0.88 (t, J=6.9 Hz, Ar(OCH₂CH₂(CH₂)₁₅CH₃)₃, 9H), 1.24-1.47 (m, Ar(OCH₂CH₂(CH₂)₁₅CH₃)₃, 90H), 1.68-1.81 (m, Ar(OCH₂CH₂(CH₂)₁₅CH₃)₃, 6H), 3.65 (s, ArCH₂CN, 2H), 3.92-3.96 (m, Ar(OCH₂CH₂(CH₂)₁₅CH₃)₃, 6H), 6.47 (s, ArH, 2H).

### 2-(3,4,5-Tris(octadecyloxy)phenyl)acetic acid) (3f)

To a suspension of nitrile 3e (2.00 g, 2.19 mmol) in methanol (150 mL) was added NaOH (30.0 g, 750 mmol) in water (20 mL). The mixture was heated to reflux for 72 h. When the reaction was cooled to rt, H₂O (100 mL) was poured to dilute the solution. The mixture was acidified by slowly adding conc. HCl to pH ˜1 and then extracted with CH₂Cl₂ (3×150 mL). After washing of the combined organic layers with brine (2×100 mL) and water (1×100 mL), the organic phase was dried over MgSO₄ and evaporated to give the crude product. The crude product was taken up in CH₂Cl₂ and then cold MeOH was added to precipitate 3f (1.80 g, 1.91 mmol, 87%). The product was used in the next step without further purification. ¹H NMR (500 MHZ, CDCl₃): δ 0.87 (t, J=7.4 Hz, Ar(OCH₂CH₂(CH₂)₁₅CH₃)₃, 9H), 1.24-1.49 (m, Ar(OCH₂CH₂(CH₃)₁₅CH₃)₃, 90H), 1.69-1.81 (m, Ar(OCH₂CH₂(CH₂)₁₅CH₃)₃, 6H), 3.48 (s, ArCH₂COOH, 2H), 3.89-3.94 (m, Ar(OCH₂CH₂(CH₂)₁₅CH₃)₃, 6H), 6.45 (s, ArH, 2H).

### Methyl-2-(3,4,5-tris(octadecyloxy)phenyl)acetate (3g)

A solution of 50% w/w BF₃ in MeOH (2.00 ml, 24.0 mmol) was added slowly under argon to a stirred solution of 3f (4.00 g, 4.38 mmol) dissolved in a mixture of MeOH (50 mL) and THF (150 mL). The mixture was heated to reflux for 48 h. After cooled to room temperature, H₂O (50 mL) was added and the mixture was extracted with CH₂Cl₂ (3×150 mL). The collected organic layers were washed with brine (1×100 mL), water (1×100 mL), dried over MgSO₄, and evaporated to obtain ester 3g as a pale yellow powder. Further purification by column chromatography with CH₂Cl:hexane (3:2), as the eluent afforded 3g (2.20 g, 2.30 mmol, 52%) as a white solid. ¹H NMR (400 MHZ, CDCl₃): δ 0.86 (t, J=6.9 Hz, Ar(OCH₂CH₂(CH₂)₁₅CH₃)₃, 9H), 1.18-1.50 (m, Ar(OCH₂CH₂(CH₂)₁₅CH₃)₃, 90H), 1.69-1.81 (m, Ar(OCH₂CH₂(CH₂)₁₅CH₃)₃, 6H), 3.51 (s, ArCH₂COOCH₃, 2H), 3.69 (s, ArCH₂COOCH₃, 3H), 3.89-3.96 (m, Ar(OCH₂CH₂(CH₂)₁₅CH₃)₃, 6H), 6.44 (s, ArH 2H).

### S2. Synthesis of the Aromatic Bidentate Thiols, R1ArDT, R2ArDT, and R3ArDT

The synthetic pathway and procedure for synthesis of R1ArDT, R2ArDT and R3ArDT are presented in Scheme S3.

### Dimethyl 2-(4-(octadecyloxy)phenyl)malonate (1b)

To a solution of 1a (2.00 g, 12.0 mmol) in THF (25 mL) was placed sodium hydride (1.73 g, 72.21 mmol) at rt under argon. The mixture was stirred for 30 min, then treated with dimethyl carbonate (30 mL), and refluxed at 90° C. for 48 h. The resultant mixture was diluted with water (50 mL), neutralized with 2 M HCl, and extracted with CH₂Cl₂ (3×100 mL). The combined organic layers were washed with water (1×100 mL), dried over MgSO₄ and evaporated under vacuum to obtain the crude product. The crude product was dissolved with a minimum volume of CH₂Cl₂ and then cold MeOH was added to the solution to precipitate 1b (4.93 g, 10.3 mmol, 86%) as a white solid. ¹H NMR (300 MHZ, CDCl₃): δ 0.86 (t, J=6.9 Hz, ArOCH₂CH₂(CH₂)₁₅CH₃, 3H), 1.25-1.44 (m, ArOCH₂CH₂(CH₂)₁₅CH₃, 30H), 1.75 (pent, J=7.8 Hz, ArOCH₂CH₂(CH₂)₁₅CH₃, 2H), 3.73 (s, Ar(CH(COOCH₃)₂), 6H), 3.92 (t, J=6.9 Hz, ArOCH₂CH₂(CH₂)₁₅CH₃, 2H), 4.57 (s, Ar (CH(COOCH₃)₂), 6.86 (d, J=8.7 Hz, ArH, 2H), 7.28 (d, J=8.7 Hz, ArH, 2H).

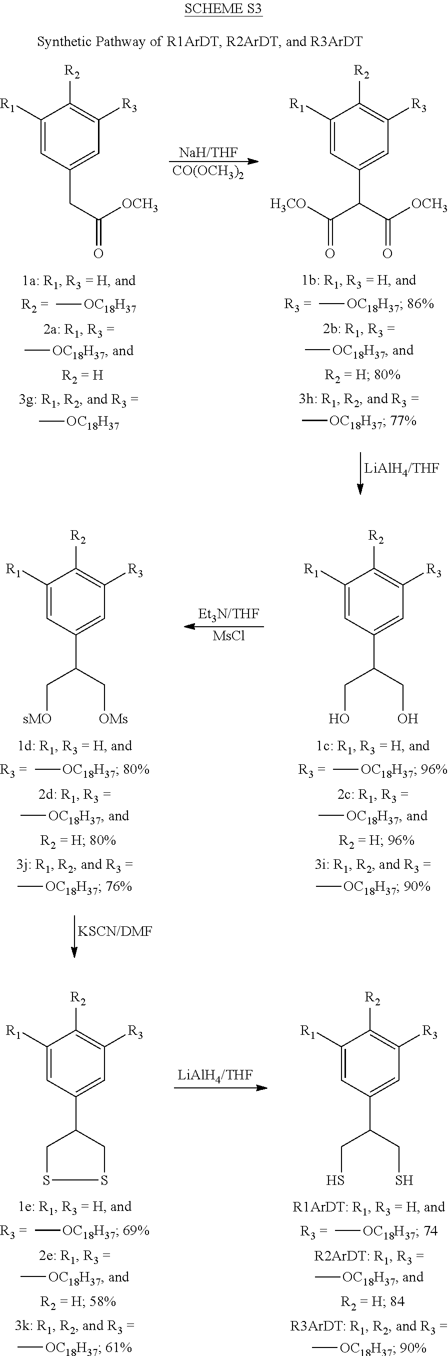

### 2-(4-(Octadecyloxy)phenyl)propane-1,3-diol (1c)

To a suspension of LiAlH₄ (0.80 g, 20 mmol) in THF (15 mL) was added slowly a solution of 1b (2.00 g, 4.19 mmol) via addition funnel under argon. The mixture was refluxed for 6 h, cooled down to rt, quenched with ethanol (25 mL) and acidified to pH ˜1 with 2 M HCl. The mixture was extracted with diethyl ether (3×150 mL). The combined organic layers were subsequently washed with a dilute HCl solution (1×100 mL), brine (1×100 mL), and water (1×100 mL), and then dried over MgSO₄. Removal of the organic solvent under vacuum gave the crude product, which was purified by co-solvent recrystallization with CH₂Cl₂ and MeOH to afford 1c (1.70 g, 4.04 mmol, 96%) as a white powder. ¹H NMR (400 MHZ, CDCl₃): δ 0.87 (t, J=6.5 Hz, ArOCH₂CH₂(CH₂)₁₅CH₃, 3H), 1.24-1.45 (m, ArOCH₂CH₂(CH₂)₁₅CH₃, 30H), 1.76 (pent, J=8.0 Hz, ArOCH₂CH₂(CH₂)₁₅CH₃, 2H), 3.15 (m (br), Ar (CH(CH₂OH)₂), 2H), 3.95 (m, ArOCH₂CH₂(CH₂)₁₅CH₃, Ar(CH(CH₂OH)₂), and Ar (CH(CH₂OH)₂), 7H), 6.89 (d, J=8.4 Hz, ArH, 2H), 7.17 (d, J=8.4 Hz, ArH, 2H).

### 2-(4-(Octadecyloxy)phenyl)propane-1,3-dimethanesulfonate (1d)

To a stirred solution of ic (2.00 g, 4.76 mmol), and triethylamine (3.98 mL, 28.6 mmol) in anhydrous THF (25 mL) was added dropwise methansulfonyl chloride (2.22 mL, 28.6 mmol) over 5 min under argon atmosphere. After the addition was completed, stirring was continued for 4 hr at rt. Ice cold water was poured into the reaction mixture to destroy any excess methansulfonyl chloride. The mixture was extracted with diethyl ether (3×100 mL). The combined organic layers were washed with dilute HCl (1×100 mL), brine (1×100 mL), and water (1×100 mL). The organic phase was dried over MgSO₄ and removed under vacuum to give the crude mesylate 1d. The crude product was dissolved with a minimum volume of CH₂Cl₂ and then MeOH was added to the solution to precipitate 1d (2.2 g, 3.82 mmol, 80%) as a white solid. ¹H NMR (300 MHZ, CDCl₃): δ 0.88 (t, J=6.9 Hz, ArOCH₂CH₂(CH₂)₁₅CH₃, 3H), 1.25-1.44 (m, ArOCH₂CH₂(CH₂)₁₅CH₃, 30H), 1.77 (pent, J=7.8 Hz, ArOCH₂CH₂(CH₂)₁₅CH₃, 2H), 2.96 (s, ArCH(CH₂SO₂CH₃)₂, 9H), 3.45 (m, ArCH(CH₂SO₂CH₃)₂, 1H), 3.93 (t, J=6.6 Hz, ArOCH₂CH₂(CH₂)₁₅CH₃, 2H), 4.50 (t, J=6.3 Hz, ArCH(CH₂SO₂CH₃)₂, 1H), 6.87 (d, J=9.0 Hz, ArH, 2H), 7.17 (d, J=9.0 Hz, ArH, 2H).

### 4-(4-(Octadecyloxy)phenyl)-1,2-dithiolane (1e)

A mixture of 1d (1.50 g, 2.60 mmol) and KSCN (5.06 g, 52.0 mmol) in a mixture solution of EtOH (10 mL) and DMF (10 mL) was stirred at 140° C. for 24 h. The resulting brownish solution was poured into cold water. The precipitate formed was filtrated, washed with water, and then dissolved in CH₂Cl₂ (250 mL). The organic layer was washed with saturated brine (1×50 mL), dried over MgSO₄ and concentrated to dryness. The crude product was purified by column chromatography on silica gel, eluting with CH₂Cl₂ to afford 1d (0.81 g, 1.79 mmol, 69%). ¹H NMR (500 MHZ, CDCl₃): δ 0.87 (t, J=6.9 Hz, ArOCH₂CH₂(CH₂)₁₅CH₃, 3H), 1.25-1.46 (m, ArOCH₂CH₂(CH₂)₁₅CH₃, 30H), 1.78 (pent, J=8.0 Hz, ArOCH₂CH₂(CH₂)₁₅CH₃, 2H), 3.26-3.42 (m, ArCH(CH₂S)₂ and ArCH(CH₂S)₂, 5H), 3.94 (t, J=6.9 Hz, ArOCH₂CH₂(CH₂)₁₅CH₃, 2H), 6.92 (d, J=8.6 Hz, ArH, 2H), 7.12 (d, J=8.6 Hz, ArH, 2H).

### 2-(4-(Octadecyloxy)phenyl)propane-1,3-dithiol (R1ArDT)

A solution of 1e (2.00 g, 4.43 mmol) in THF (25 ml) was added dropwise into a suspension solution of LiAlH₄ (0.84 g, 22 mmol) in THF (10 mL) through an addition funnel argon. The mixture was stirred at rt for 6 h, quenched with ethanol (25 mL), then acidified with 2 M HCl (previously degas by bubbling with argon). After stirred for 10 min, the mixture was extracted with CH₂Cl₂ (3×100 mL). The combined organic layers were washed with brine (1×100 mL) and water (1×100 mL), dried over MgSO₄, and evaporated to dryness to give R1ArDT (1.50 g, 3.31 mmol, 74%). ¹H NMR (500 MHZ, CDCl₃): δ 0.87 (t, J=6.9 Hz, ArOCH₂CH₂(CH₂)₁₅CH₃, 3H), 1.25-1.48 (m, ArOCH₂CH₂(CH₂)₁₅CH₃, and ArCH(CH₂SH)₂, 32H), 1.76 (pent, J=8.0 Hz, ArOCH₂CH₂(CH₂)₁₅CH₃, 2H), 2.84-2.87 (m, ArCH(CH₂SH)₂ and ArCH(CH₂SH)₂, 5H), 3.93 (t, J=6.9 Hz, ArOCH₂CH₂(CH₂)₁₅CH₃, 2H), 6.89 (d, J=8.6 Hz, ArH, 2H), 7.14 (d, J=8.6 Hz, ArH, 2H). ¹³C NMR (125 MHZ, CDCl₃): δ 14.22, 22.79, 26.16, 29.53, 29.70, 32.02, 51.38, 68.07, 114.71, 128.90, 132.98, 158.92.

### Dimethyl 2-(3,5-bis(octadecyloxy)phenyl)malonate (2b)

Following the procedure described for 1b. To a reaction mixture of 2a (1.00 g, 1.45 mmol) and sodium hydride (0.21 g, 8.73 mmol) in THF (20 mL) was added dropwise dimethyl carbonate (30 mL) to obtain diester 2b (0.87 g, 1.16 mmol, 80%) as a white powder product. ¹H NMR (500 MHZ, CDCl₃): δ 0.87 (t, J=6.9 Hz, Ar(OCH₂CH₂(CH₂)₁₅CH₃)₂, 6H), 1.24-1.43 (m, Ar(OCH₂CH₂(CH₂)₁₅CH₃)₂, 60H), 1.74 (pent, J=8.0 Hz, Ar(OCH₂CH₂(CH₂)₁₅CH₃)₂, 4H), 3.74 (s, ArCH(CH₂COCH₃)₂, 6H), 3.91 (t, J=6.3 Hz, Ar(OCH₂CH₂(CH₂)₁₅CH₃)₂, 4H), 4.54 (s, ArCH(COOCH₃)₂, 1H), 6.40 (s, ArH, 1H), 6.51 (s, ArH, 2H).

### 2-(3,5-Bis(octadecyloxy)phenyl)propane-1,3-diol (2c)

Following the procedure described for 1c. To a suspension of LiAlH₄ (0.38 g, 10 mmol) in THF (10 mL) was added a solution of 2b (1.50 g, 2.01 mmol) in THF (15 mL) to obtain 2c (1.34 g, 1.94 mmol, 96%) as a white powder. ¹H NMR (400 MHZ, CDCl₃): δ 0.87 (t, J=6.8 Hz, Ar(OCH₂CH₂(CH₂)₁₅CH₃)₂, 6H), 1.24-1.43 (m, Ar(OCH₂CH₂(CH₂)₁₅CH₃)₂, 60H), 1.74 (pent, J=7.8 Hz, Ar(OCH₂CH₂(CH₂)₁₅CH₃)₂, 4H), 3.05 (br, Ar (CH(CH₂OH)₂), 2H), 3.90-3.95 (m, ArCH(CH₂OH)₂, ArCH(CH₂OH)₂, and Ar(OCH₂CH₂(CH₂)₁₅CH)₂, 6H), 6.34 (s, ArH, 2H).

### 2-(3,5-Bis(octadecyloxy)phenyl)propane-1,3-diyl dimethanesulfonate (2d)

Following the procedure described for 1d. A mixture of 2c (1.50 g, 2.17 mmol) and triethylamine (1.82 mL, 13.1 mmol) in THF (30 mL) was treated with methansulfonyl chloride (1.02 mL, 13.1 mmol) to obtain the crude mesylate 2d (1.48 g, 1.75 mmol, 80%) as a white powder product. The crude product was directly used in the next step without any further purification. ¹H NMR (500 MHZ, CDCl₃): δ 0.87 (t, J=6.8 Hz, Ar(OCH₂CH₂(CH₂)₁₅CH₃)₂, 6H), 1.25-1.49 (m, Ar(OCH₂CH₂(CH₂)₁₅CH₃)₂, 60H), 1.77 (pent, J=7.4 Hz, Ar(OCH₂CH₂(CH₂)₁₅CH₃)₂, 4H), 2.97 (s, ArCH(CH₂SO₂CH₃)₂, 6H), 3.36 (m, ArCH(CH₂SO₂CH₃)₂, 1H), 3.90 (t, J=6.9 Hz, ArO(CH₂CH₂(CH₂)₁₅CH₃)₂, 4H), 4.54-4.49 (m, ArCH(CH₂SO₂CH₃)₂, 4H), 6.34 (s, ArH, 2H), 6.39 (s, ArH, 1H).

### 4-(3,5-Bis(octadecyloxy)phenyl)-1,2-dithiolane (2e)

Following the procedure described for 1e. The mesylate 2d (1.50 g, 1.77 mmol) was treated with KSCN (3.45 g, 35.5 mmol) in a mixture solution of EtOH (10 mL) and DMF (10 mL). The crude product was purified by column chromatography using CH₂Cl₂:hexanes (3:2) as the eluent to afford 2e (0.74 g, 1.0 mmol, 58%). ¹H NMR (300 MHZ, CDCl₃): δ 0.87 (t, J=7.3 Hz, Ar(OCH₂CH₂(CH₂)₁₅CH₃)₂, 6H), 1.25-1.49 (m, Ar(OCH₂CH₂(CH₂)₁₅CH₃)₂, 60H), 1.77 (pent, J=8.2 Hz, Ar(OCH₂CH₂(CH₂)₁₅CH₃)₂, 4H), 3.26-3.43 (m, ArCH(CH₂S)₂, and ArCH(CH₂S)₂, 5H), 3.90 (t, J=6.9 Hz, ArO(CH₂CH₂(CH₂)₁₅CH₃)₂, 4H), 6.31 (s, ArH, 2H), 6.42 (s, ArH, 1H).

### 2-(3,5-Bis(octadecyloxy)phenyl)propane-1,3-dithiol (R2ArDT)

Following the procedure described for R1ArDT. To a suspension of LiAlH₄ (0.13 g, 3.5 mmol) in THF (10 mL) was added dropwise a solution of 2e (0.50 g, 0.70 mmol) in THF (15 mL) to yield the R2ArDT (0.42 g, 0.58 mmol, 84%). ¹H NMR (500 MHZ, CDCl₃): δ 0.87 (t, J=6.9 Hz, Ar(OCH₂CH₂(CH₂)₁₅CH₃)₂, 6H), 1.25-1.49 (m, Ar(OCH₂CH₂(CH₂)₁₅CH₃)₂ and ArCH(CH₂SH)₂, 62H), 1.77 (pent, J=8.0 Hz, Ar(OCH₂CH₂(CH₂)₁₅CH₃)₂, 4H), 2.85 (m, ArCH(CH₂SH)₂ and ArCH(CH₂SH)₂, 5H), 3.90 (t, J=6.3 Hz, ArO(CH₂CH₂(CH₂)₁₅CH₃)₂, 4H), 6.30 (s, ArH, 2H), 6.34 (s, ArH, 1H). ¹³C NMR (125 MHZ, CDCl₃): δ 14.22, 22.79, 26.16, 29.52, 29.71, 32.02, 52.66, 68.14, 99.80, 106.54, 144.52, 160.58.

### Dimethyl 2-(3,4,5-tris(octadecyloxy)phenyl)malonate (3h)

Following the reaction described for 1b. To a reaction mixture of 3g (2.00 g, 2.09 mmol) and sodium hydride (0.30 g, 12.6 mmol) in THF (20 mL) was added dropwise dimethyl carbonate (30 mL) to obtain 3h (1.64 g, 1.62 mmol, 77%). ¹H NMR (500 MHZ, CDCl₃): δ 0.87 (t, J=6.9 Hz, Ar(OCH₂CH₂(CH₂)₁₅CH₃)₃, 9H), 1.18-1.49 (m, Ar(OCH₂CH₂(CH₂)₁₅CH₃)₃, 90H), 1.69-1.83 (m, Ar(OCH₂CH₂(CH₂)₁₅CH₃)₃, 6H), 3.75 (s, ArCH(CH₂COCH₃)₂, 6H), 3.91-3.96 (m, Ar(OCH₂CH₂(CH₂)₁₅CH₃)₂, 6H), 4.51 (s, ArCH(COOCH₃)₂, 1H), 6.56 (s, ArH, 2H).

### 2-(3,4,5-Tris(octadecyloxy)phenyl)propane-1,3-diol (31)

Following the reaction described for 1c. To a suspension of LiAlH₄ (0.28 g, 7.4 mmol) in THF (10 mL) was added a solution of 3h (1.50 g, 1.48 mmol) in THF (15 mL) to obtain 31 (1.28 g, 1.33 mmol, 90%). ¹H NMR (500 MHZ, CDCl₃): δ 0.87 (t, J=6.9 Hz, Ar(OCH₂CH₂(CH₂)₁₅CH₃)₃, 9H), 1.25-1.49 (m, Ar(OCH₂CH₂(CH₂)₁₅CH₃)₂, 90H), 1.69-1.80 (m, Ar(OCH₂CH₂(CH₂)₁₅CH₃)₃, 6H), 3.81-3.93 (m, ArCH(CH₂OH)₂, ArCH(CH₂OH)₂, Ar(OCH₂CH₂(CH₂)₁₅CH₃)₃, 9H), 6.39 (s, ArH, 2H).

### 2-(3,4,5-Tris(octadecyloxy)phenyl)propane-1,3-diyl dimethanesulfonate (3j)

Following the procedure described for 1d. A mixture of 31 (1.00 g, 1.04 mmol) and triethylamine (0.87 mL, 6.3 mmol) in THF (30 mL) was treated with methansulfonyl chloride (0.49 mL, 6.3 mmol) to obtain the crude mesylate 3j (0.89 g, 0.80 mmol, 76%) as a white powder product. The crude product was directly used in the next step without any further purification. ¹H NMR (500 MHZ, CDCl₃): δ 0.87 (t, J=7.4 Hz, Ar(OCH₂CH₂(CH₂)₁₅CH₃)₃, 9H), 1.25-1.49 (m, Ar(OCH₂CH₂(CH₂)₁₅CH₃)₃, 90H), 1.70-1.80 (m, Ar(OCH₂CH₂(CH₂)₁₅CH₃)₃, 6H), 2.95 (s, ArCH(CH₂SO₂CH₃)₂, 6H), 3.35 (m, ArCH(CH₂SO₂CH₃)₂, 1H), 3.90-3.94 (m, ArO(CH₂CH₂(CH₂)₁₅CH₃)₃, 6H), 4.54-4.47 (m, ArCH(CH₂SO₂CH₃)₂, 4H), 6.40 (s, ArH, 2H).

### 4-(3,4,5-Tris(octadecyloxy)phenyl)-1,2-dithiolane (3k)

Following the procedure described for 1e. The mesylate 3j (1.00 g, 0.90 mmol) was treated with KSCN (1.75 g, 18.0 mmol) in a solution mixture of EtOH (10 mL) and DMF (10 mL). The crude product was purified by column chromatography using hexanes:EtOAc (4:1) as the eluent to afford 3k (0.54 g, 0.55 mmol, 61%). ¹H NMR (400 MHZ, CDCl₃): δ 0.87 (t, J=6.9 Hz, Ar(OCH₂CH₂(CH₂)₁₅CH₃)₃, 9H), 1.25-1.49 (m, Ar(OCH₂CH₂(CH₂)₁₅CH₃)₃, 90H), 1.70-1.80 (m, Ar(OCH₂CH₂(CH₂)₁₅CH₃)₃, 6H), 3.27-3.40 (m, ArCH(CH₂S)₂ and ArCH(CH₂S)₂, 5H), 3.90-3.94 (m, ArO(CH₂CH₂(CH₂)₁₅CH₃)₃, 6H), 6.34 (s, ArH, 2H).

### 2-(3,5-Bis(octadecyloxy)phenyl)propane-1,3-dithiol (R3ArDT)

Following the procedure described for R1ArDT. To a suspension of LiAlH₄ (0.04 g, 1.01 mmol) in THF (10 mL) was added dropwise a solution of 3k (0.20 g, 0.20 mmol) in THF (15 mL) to yield R3ArDT (0.18 g, 0.18 mmol, 90%). ¹H NMR (500 MHZ, CDCl₃): δ 0.87 (t, J=6.3 Hz, Ar(OCH₂CH₂(CH₂)₁₅CH₃)₃, 9H), 1.25-1.48 (m, Ar(OCH₂CH₂(CH₂)₁₅CH₃)₃ and ArCH(CH₂SH)₂, 90H), 1.70-1.80 (m, Ar(OCH₂CH₂(CH₂)₁₅CH₃)₃, 6H), 2.83-2.85 (m, ArCH(CH₂SH)₂ and ArCH(CH₂S)₂, 5H), 3.91-3.94 (m, ArO(CH₂CH₂(CH₂)₁₅CH₃)₃, 6H), 6.33 (s, ArH, 2H). ¹³C NMR (125 MHZ, CDCl₃): δ 14.24, 22.80, 26.21, 29.48, 29.56, 29.76, 29.83, 30.44, 32.03, 52.46, 69.31, 73.47, 106.40, 136.19, 137.48, 153.30.

### S3. Synthesis of 4,6-Bis(octadecyloxy)-1,3-phenylene)dimethanethiol (R1ArmDT)

The synthetic pathway and procedure for synthesis of (4,6-bis(octadecyloxy)-1,3-phenylene)dimethanethiol, R1ArmDT are presented in Scheme S4.

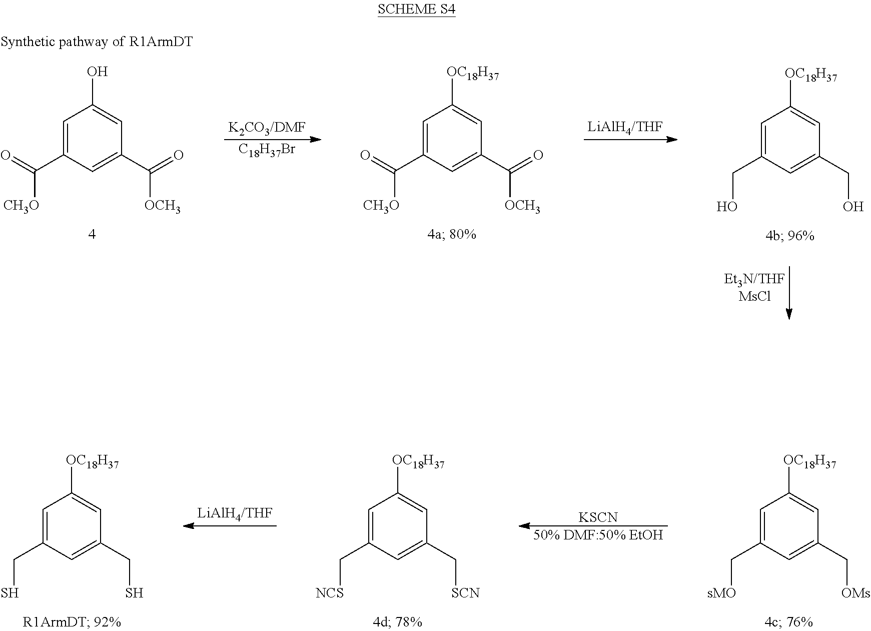

### Dimethyl 5-(octadecyloxy)isophthalate (4a)

A mixture of 1-bromoctadecane (4.12 g, 12.37 mmol), dimethyl 5-hydroxyisophthalate (2.00 g, 9.52 mmol), and K₂CO₃ (2.63 g, 19.0 mmol) in DMF (150 mL) was stirred at 120° C. for overnight. After cooling, K₂CO₃ was removed by filtration, and then the filtrate was diluted with H₂O and acidified with 2 M HCl. The aqueous layer was extracted with CH₂Cl₂ (3×250 mL). The organic layers were combined and washed with H₂O (3×50 mL), dried over MgSO₄, and concentrated to dryness to afford the crude product. The crude product was chromatographed on silica gel using a mixture of CH₂Cl₂ and hexane (3:2) as the eluent to give 4a (3.52 g, 7.61 mmol, 80%) as a white powder product. ¹H NMR (500 MHZ, CDCl₃): δ 0.87 (t, J=6.9 Hz, ArOCH₂CH₂(CH₂)₁₅CH₃, 3H), 1.26-1.46 (m, ArOCH₂CH₂(CH₂)₁₅CH₃, 30H), 1.80 (pent, J=6.9 Hz, ArOCH₂CH₂(CH₂)₁₅CH₃, 2H), 3.94 (s, Ar(COOCH₃)₂, 6H), 4.02 (t, J=6.9 Hz, ArOCH₂CH₂(CH₂)₁₅CH₃, 2H), 7.73 (s, ArH, 2H), 8.25 (ArH, 2H).

### (5-(Octadecyloxy)-1,3-phenylene)dimethanol (4b)

A solution of 4a (3.00 g, 6.48 mmol) in THF (20 mL) was added dropwise to a suspension of LiAlH₄ (0.62 g, 16 mmol) in THF (25 mL) under argon. The reaction was stirred and heated to reflux for 6 h, then quenched with H₂O, and acidified with 2 M HCl. The mixture was extracted with diethyl ether (3×150 mL) The combined organic layers were washed with brine (1×100 mL) and water (1×100 mL), dried over MgSO₄, and evaporated to dryness. The crude product was taken up in CH₂Cl₂ and then cold MeOH was added to precipitate 4b (2.54 g, 6.25 mmol, 96%) as white solid. ¹H NMR (500 MHZ, CDCl₃): δ 0.87 (t, J=6.9 Hz, ArOCH₂CH₂(CH₂)₁₅CH₃, 3H), 1.26-1.45 (m, ArOCH₂CH₂(CH₂)₁₅CH₃, 30H), 1.63 (t, J=4.6 Hz, Ar(CH₂OH)₂, 2H) 1.77 (pent, J=7.4 Hz, ArOCH₂CH₂(CH₂)₁₅CH₃, 2H), 3.96 (t, J=6.9 Hz, ArOCH₂CH₂(CH₂)₁₅CH₃, 2H), 4.66 (d, J=4.6 Hz, Ar(CH₂OH)₂, 4H), 6.81 (s, ArH, 2H), 6.93 (ArH, 2H).

### (5-(Octadecyloxy)-1,3-phenylene)bis(methylene) dimethanesulfonate (4c)

Methansulfonyl chloride (1.15 mL, 14.8 mmol) was added dropwise over 5 min to a stirred solution of 4b (2.00 g, 4.92 mmol) and triethylamine (2.06 mL, 14.8 mmol) in anhydrous THF (25 mL) under argon. The mixture was stirred for 4 h at rt. To destroy excess methansulfonyl chloride, ice-cold water was added to the reaction flask. The mixture was extracted with diethyl ether (3×100 mL). The organic layers were washed successively with 2M HCl (1×100 mL) and water (1×100 mL), dried over MgSO₄, and concentrated to dryness to yield the crude mesylate 4c. The crude product was dissolved with a minimum volume of CH₂Cl₂ and cold MeOH was added to precipitate 4c (2.19 g, 3.73 mmol, 76%). ¹H NMR (400 MHZ, CDCl₃): δ 0.87 (t, J=6.9 Hz, ArOCH₂CH₂(CH₂)₁₅CH₃, 3H), 1.25-1.45 (m, ArOCH₂CH₂(CH₂)₁₅CH₃, 30H), 1.77 (pent, J=6.9 Hz, ArOCH₂CH₂(CH₂)₁₅CH₃, 2H), 2.97 (s, Ar(CH₂SO₂CH₃)₂, 6H), 3.96 (t, J=6.4 Hz, ArOCH₂CH₂(CH₂)₁₅CH₃, 2H), 5.19 (s, J=4.6 Hz, Ar(CH₂SO₂CH)₂, 4H), 6.94 (s, ArH, 2H), 7.00 (s, ArH, 2H).

### 1-(Octadecyloxy)-3,5-bis(thiocyanatomethyl)benzene (4d)

A mixture of dimesylate 4c (2.00 g, 3.55 mmol) and KSCN (3.45 g, 35.53 mmol) in a mixture of ethanol (15 mL) and DMF (15 mL) was stirred at 140° C. for 24 h under argon. The mixture was poured into cold water to precipitate the dithiocyanate crude product 4d. The crude product was washed several times with H₂O and purified by column chromatography on silica gel eluting with CH₂Cl₂ to obtain 4d (1.35 g, 2.76 mmol, 78%). ¹H NMR (400 MHZ, CDCl₃): δ 0.87 (t, J=6.9 Hz, ArOCH₂CH₂(CH₂)₁₅CH₃, 3H), 1.25-1.45 (m, ArOCH₂CH₂(CH₂)₁₅CH₃, 30H), 1.78 (pent, J=6.9 Hz, ArOCH₂CH₂(CH₂)₁₅CH₃, 2H), 3.97 (t, J=6.4 Hz, ArOCH₂CH₂(CH₂)₁₅CH₃, 2H), 4.10 (s, Ar(CH₂SCN)₂, 6H), 6.94 (s, ArH, 2H), 7.00 (s, ArH, 2H).

### (5-(Octadecyloxy)-1,3-phenylene)dimethanethiol (R1ArmDT)

A solution of 4d (1.00 g, 2.05 mmol) in THF (20 mL) was added dropwise to a suspension of LiAlH₄ (0.19 g, 5.12 mmol) in THF (15 mL) under argon. The reaction was stirred at rt for 6 h, then quenched with H₂O, and acidified with 2 M HCl. The mixture was extracted with CH₂Cl₂ (3×100 mL). The combined organic layers were washed with brine (1×100 mL) and water (1×100 mL), dried over MgSO₄, and evaporated to dryness to give R1ArmDT (0.83 g, 1.89 mmol, 92%). ¹H NMR (500 MHZ, CDCl₃): δ 0.87 (t, J=6.9 Hz, ArOCH₂CH₂(CH₂)₁₅CH₃, 3H), 1.25-1.45 (m, ArOCH₂CH₂(CH₂)₁₅CH₃ and Ar(CH₂SH)₂, 32H), 1.76 (pent, J=6.9 Hz, ArOCH₂CH₂(CH₂)₁₅CH₃, 2H), 3.68 (d, J=7.5 Hz, Ar(CH₂SH)₂, 4H), 3.94 (t, J=6.3 Hz, ArOCH₂CH₂(CH₂)₁₅CH₃, 2H), 6.74 (s, ArH, 2H), 6.84 (s, ArH, 2H). ¹³CNMR (125 MHZ, CDCl₃): d 14.22, 22.78, 26.15, 29.01, 29.36, 29.46, 29.79, 32.02, 68.13, 112.98, 119.88, 142.99, 159.72.

### S4. Synthesis of R1ArTT

The synthetic pathway used to prepare the aromatic tridentate adsorbate, 2-(mercaptomethyl)-2-(4-(octadecyloxy)phenyl)propane-1,3-dithiol (R1ArTT) is shown in Scheme S5.

### 3-Hydroxy-2-(hydroxymethyl)-2-(4-(octadecyloxy)phenyl)propanoic acid (5a)

Sodium methoxide (5.5 g, 0.10 mol) and paraformadehyde (10.0 g, 0.26 mol) were added to a stirred solution of 1a (5.00 g, 12.8 mmol) in DMF (100 mL). The mixture was stirred at rt for 120 h, then poured into a mixture of ice-water, and acidified to pH ˜1 with 2 M HCl. The mixture was extracted with diethyl ether (3200 mL). The combined organic layers were washed with brine (1×100) and water (1×100 mL), dried over MgSO₄, and concentrated to dryness to obtain the crude product 5c. The crude product was tritulated several times with CH₂Cl₂ and hexane to give the pure product 5a (2.13 g, 4.59 mmol, 36%) as a white powder. ¹H NMR (500 MHZ, Acetone-d6): δ 0.91 (t, J=7.4 Hz, ArOCH₂CH₂(CH₂)₁₅CH₃, 3H), 1.26-1.45 (m, ArOCH₂CH₂(CH₂)₁₅CH₃, 30H), 1.73 (pent, J=6.9 Hz, ArOCH₂CH₂(CH₂)₁₅CH₃, 2H), 3.94 (t, J=6.9 Hz, ArOCH₂CH₂(CH₂)₁₅CH₃, 2H), 4.11-4.26 (dd, J=45.8, 10.3 Hz, ArC(CH₂OH)₂, 4H), 6.85 (d, J=8.7 Hz, ArH, 2H), 7.25 (d, J=8.7 Hz, ArH, 2H).

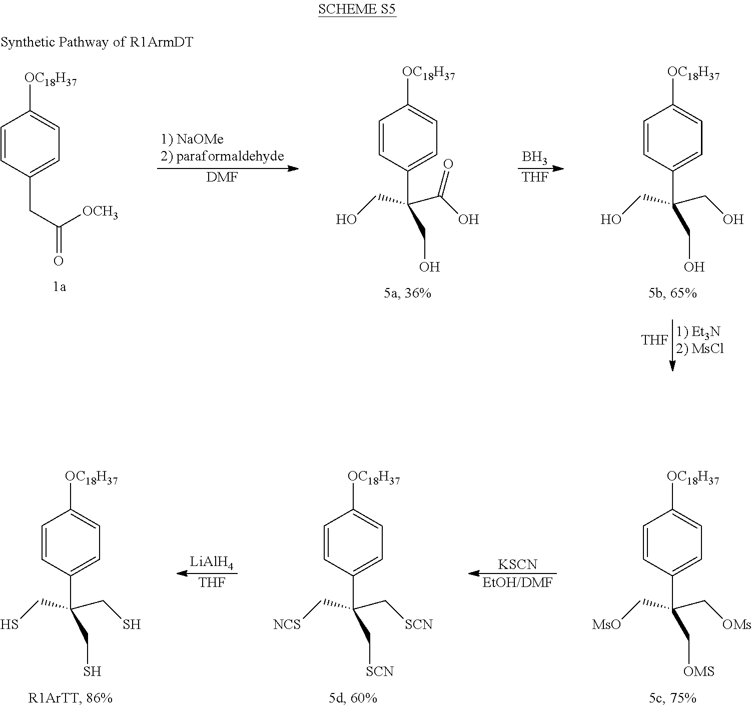

### 2-(Hydroxymethyl)-2-(4-(octadecyloxy)phenyl)propane-1,3-diol (5b)

1 M BH₃ (43.07 ml, 43.07 mmol) was added dropwise to a solution of 5a (4.00 g, 8.61 mmol) in dry THF (50 mL). The mixture was stirred for 2 hours at 0° C. and warmed to room temperature for overnight. The reaction was quenched with water (100 mL) and extracted with diethyl ether (3×150 mL). The combined organic layers were washed subsequently with brine (1×100 mL) and water (1×100 mL), dried over MgSO₄ and concentrated to dryness to obtain the crude product. The crude product was recrystallized in EtOAC to obtain triol 5b (2.53 g, 5.62 mmol, 65%) as a white powder. ¹H NMR (500 MHZ, Acetone-d6): δ 0.85 (t, J=6.9 Hz, ArOCH₂CH₂(CH₂)₁₅CH₃, 3H), 1.26-1.48 (m, ArOCH₂CH₂(CH₂)₁₅CH₃, 30H), 1.73 (pent, J=6.9 Hz, ArOCH₂CH₂(CH₂)₁₅CH₃, 2H), 2.79 (d, J=17.2, ArC(CH₂OH)₃, 3H), 3.94 (s, ArOCH₂CH₂(CH₂)₁₅CH₃, 6H), 6.81 (d, J=9.2 Hz, ArH, 2H), 7.34 (d, J=9.2 Hz, ArH, 2H).

### 2-(Methanesulonyl-methyl)-2-(4-(octadecyloxy)phenyl)propane-1,3-dimethanesulfonate (5c)

To the stirred solution of 5b (2.00 g, 4.44 mmol) and triethylamine (5.57 mL, 40.0 mmol) in dry THF (25 mL) was added dropwise methansulfonyl chloride (3.10 mL, 40.0 mmol) over 5 min. Stirring of the mixture was continued at rt for 4 h under argon. The mixture of ice-cold water was poured into the reaction flask to destroy any remaining methansulfonyl chloride. The mixture was extracted with diethyl ether (3×150 mL). The combined organic phases were washed with 2 M HCl (1×100 mL) and water (1×100 mL), dried over MgSO₄, and concentrated to dryness to obtain the crude of trimesylate 5c. Further purification by co-solvent recrytallization with CH₂Cl₂ and MeOH afforded the pure product 5c (2.27 g, 3.31 mmol, 75%) as a white powder. ¹H NMR (500 MHZ, CDCl₃): δ 0.87 (t, J=6.9 Hz, ArOCH₂CH₂(CH₂)₁₅CH₃, 3H), 1.25-1.45 (m, ArOCH₂CH₂(CH₂)₁₅CH₃, 30H), 1.77 (pent, J=6.9 Hz, ArOCH₂CH₂(CH₂)₁₅CH₃, 2H), 2.98 (s, ArC(CH₂)₃(SO₂CH₃)₃, 9H), 3.94 (t, J=6.9 Hz, ArOCH₂CH₂(CH₂)₁₅CH₃, 2H), 4.54 (s, ArC(CH₃)₃(SO₂CH₃)₃, 6H), 6.92 (d, J=9.2 Hz, ArH, 2H), 7.23 (d, J=9.2 Hz, ArH, 2H).

### 1-(1,3-Dithiocyanato-2-(thiocyanatomethyl)propan-2-yl)-4(octadeclyoxy)benzene (5d)

A mixture of trimesylate 5c (2.00 g, 2.92 mmol) and KSCN (8.51 g, 87.6 mmol) in a solution of EtOH (15 mL) and DMF (15 mL) was stirred at 140° C. for 24 h. The resulting brownish solution was poured into cold water. The precipitate formed was filtrated, wash with water, and then dissolved in CH₂Cl₂. The organic layer was washed with water (1×100 mL) and brine (1×100 mL), dried over MgSO₄, and concentrated to dryness. The crude product was purified by column chromatography on silica gel eluting with a mixture of CH₂Cl₂:hexane (3:2) to afford 5d (1.00 g, 1.74 mmol, 60%). ¹H NMR (400 MHZ, CDCl₃): δ 0.87 (t, J=6.3 Hz, ArOCH₂CH₂(CH₂)₁₅CH₃, 3H), 1.22-1.43 (m, ArOCH₂CH₂(CH₂)₁₅CH₃, 30H), 1.75 (pent, J=6.9 Hz, ArOCH₂CH₂(CH₂)₁₅CH₃, 2H), 3.68 (s, Ar(CH₂SCN)₃, 6H), 3.95 (t, J=6.3 Hz, ArOCH₂CH₂(CH₂)₁₅CH₃, 2H), 6.96 (d, J=8.0 Hz, ArH, 2H), 7.12 (d, J=8.0 Hz, ArH, 2H).

### 2-(Mercaptomethyl)-2-(4-(octadecyloxy)phenyl)propane-1,3-dithiol (R1ArTT)

To a suspension of LiAlH₄ (0.10 g, 2.6 mmol) in THF (25 mL) was add dropwise a solution of 5d (0.20 g, 0.35 mmol) in THF. The reaction was stirred at rt for 6 h, and then quenched under argon with distilled water (25 mL), and acidified with conc. HCl. After stirred for 10 min, the mixture was extracted with CH₂Cl₂ (3×100 mL). The combined organic layers were washed with brine (1×50 mL) and water (1×50 mL), dried over MgSO₄, and evaporated to dryness to give the crude product. The crude product was purified by column chromatography on silica gel eluting with a mixture of CH₂Cl₂ and hexame (3:2) to afford R1ArTT (0.15 g, 0.30 mmol, 86%). ¹H NMR (500 MHZ, CDCl₃): δ 0.88 (t, J=8.7 Hz, ArOCH₂CH₂(CH₂)₁₅CH₃, 3H), 1.02 (t, J=8.6 Hz, Ar(CH₂SH)₃, 3H), 1.24-1.48 (m, ArOCH₂CH₂(CH₂)₁₅CH₃, 30H), 1.76 (pent, J=8.7 Hz, ArOCH₂CH₂(CH₂)₁₅CH₃, 2H), 3.05 (d, J=8.6 Hz, Ar(CH₂SH)₃, 6H), 3.94 (t, J=8.3 Hz, ArOCH₂CH₂(CH₂)₁₅CH₃, 2H), 6.89 (d, J=8.6 Hz, ArH, 2H), 7.14 (d, J=8.6 Hz, ArH 2H). ¹³C NMR (125 MHZ, CDCl₃): d 14.23, 22.79, 26.17, 29.46, 29.70, 31.03, 32.02, 47.48, 68.05, 114.68, 128.09, 132.21, 158.13.

### S5. Synthesis of the Aromatic Monodentate Thiols, R1ArMT, R2ArMT, and R3ArMT

The synthetic pathway and procedure for synthesis of R1ArMT, R2ArMT, and R3ArMT is illustrated in Scheme 6. The intermediate compounds 1a, 2a, and 3g are used to prepare R1ArMT, R2ArMT, and R3ArMT, respectively.

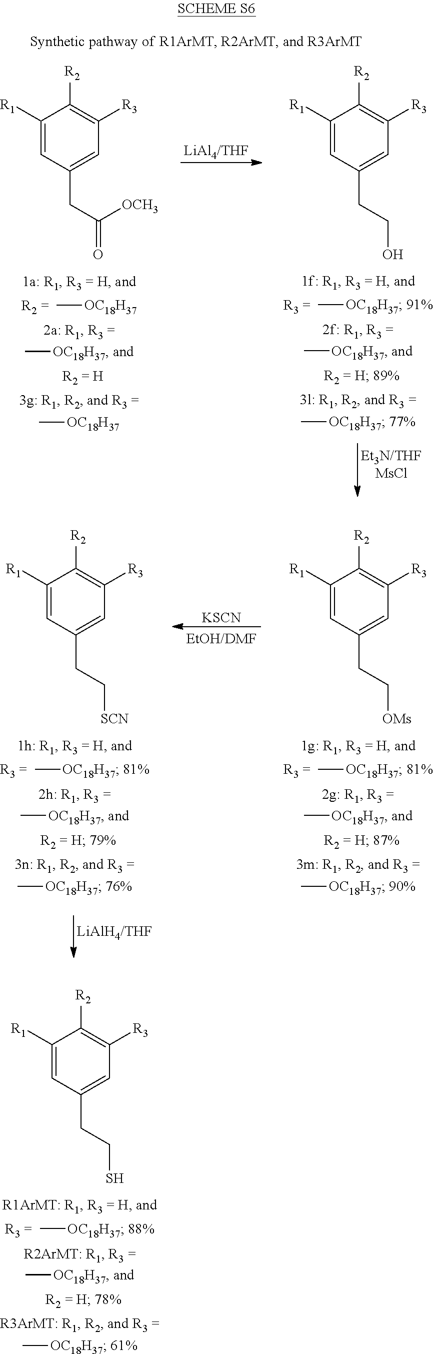

### 2-(4-(Octadecyloxy)phenyl)ethanol (1f)

To a suspension of LiAlH₄ (0.45 g, 12 mmol) in THF (25 mL) was added dropwise a solution of 1a (2.00 g, 4.78 mmol) in THF (20 mL). The mixture was refluxed for 6 h under argon, quenched with water, and acidified with 2 M HCl. After being stirred for 10 min, the resultant mixture was extracted with CH₂Cl₂ (3×150 mL). The combined organic layers were washed subsequently with brine (3×50 mL) and water (3×50 mL), dried over MgSO₄, and evaporated to dryness to give (1.70 g, 4.35 mmol, 91%) of 1c. ¹H NMR (500 MHZ, CDCl₃): δ 0.87 (t, J=6.9 Hz, ArOCH₂CH₂(CH₂)₁₅CH₃, 3H), 1.25-1.47 (m, ArOCH₂CH₂(CH₂)₁₅CH₃, 30H), 1.76 (pent, J=7.4 Hz, ArOCH₂CH₂(CH₂)₁₅CH₃, 2H), 2.80 (t, J=6.9 Hz, ArCH₂CH₂OH), 3.64 (t, J=6.9 Hz, ArCH₂CH₂OH, 2H), 3.81 (t, J=6.9 Hz, ArCH₂CH₂OH), 1H), 3.92 (t, J=7.4 Hz, ArOCH₂CH₂(CH₂)₁₅CH₃, 2H), 6.84 (d, J=8.6 Hz, ArH, 2H), 7.12 (d, J=8.6 Hz, ArH, 2H).

### 4-(Octadecyloxy)phenethylmethanesulfonate (1g)

To a stirred solution of 1f (2.00 g, 4.75 mmol) and triethylamine (1.99 ml, 14.3 mmol) in anhydrous THF (25 mL) was added dropwise methansulfonyl chloride (1.11 mL, 14.3 mmol) over 5 min. Stirring of the mixture was continued at rt for 4 h under argon. Ice-cold water was poured into the reaction flask to destroy any remaining methanesulfonyl chloride. The mixture was extracted with diethyl ether (3×100 mL). The combined organic phase were washed successively with 2 M HCl (1×100 mL) and water (1×100 mL), dried over MgSO₄, and concentrated to dryness. A minimum volume of CH₂Cl₂ was used to dissolve the crude product, and then MeOH was added to the solution to obtain the crude mesylate 1g (1.80 g, 3.84 mmol, 81%). The crude product was used in the next step without any purification. ¹H NMR (500 MHZ, CDCl₃): δ 0.87 (t, J=6.9 Hz, ArOCH₂CH₂(CH₂)₁₅CH₃, 3H), 1.25-1.47 (m, ArOCH₂CH₂(CH₂)₁₅CH₃, 30H), 1.76 (pent, J=7.8 Hz, ArOCH₂CH₂(CH₂)₁₅CH₃, 2H), 2.84 (s, ArCH₂CH₂SO₂CH₃, 3H), 2.98 (t, J=6.9 Hz, ArCH₂CH₂SO₂CH₃, 2H), 3.92 (t, J=6.9 Hz, ArOCH₂CH₂(CH₂)₁₅CH₃, 2H), 4.37 (t, J=6.9 Hz, ArCH₂CH₂SO₂CH₃, 2H), 6.83 (d, J=8.7 Hz, ArH, 2H), 7.12 (d, J=8.7 Hz, ArH, 2H).

### 1-(Octadecyloxy)-4-(2-thiocyanatethyl)benzene (1h)

A mixture of 1g (1.00 g, 2.13 mmol) and KSCN (2.07 g, 21.3 mmol) in the mixture of EtOH (10 mL) and DMF (10 mL) was stirred at 140° C. for 24 h. The resulting mixture was poured into cold water. The precipitate formed was filtered, washed with water, and then dissolved in CH₂Cl₂ (250 mL). The organic layer was washed with saturated brine (1×50 mL), dried over MgSO₄, and concentrated to dryness. The crude product was purified by column chromatography on silica gel eluting with CH₂Cl₂ to afford 1e (0.75 g, 1.7 mmol, 81%). ¹H NMR (500 MHZ, CDCl₃): δ 0.87 (t, J=6.9 Hz, ArOCH₂CH₂(CH₂)₁₅CH₃, 3H), 1.20-1.48 (m, ArOCH₂CH₂(CH₂)₁₅CH₃, 30H), 1.76 (pent, J=6.9 Hz, ArOCH₂CH₂(CH₂)₁₅CH₃, 2H), 3.05 (t, J=7.4 Hz, ArCH₂CH₂SCN), 3.13 (t, J=7.4 Hz, ArCH₂CH₂SCN, 2H), 3.92 (t, J=6.9 Hz, ArOCH₂CH₂(CH₂)₁₅CH₃, 2H), 6.84 (d, J=8.6 Hz, ArH, 2H), 7.10 (d, J=8.6 Hz, ArH, 2H).

### 2-(4-Octadecyloxy)phenyl)etanethiol (R1ArMT)

To a suspension LiAlH₄ (0.22 g, 5.8 mmol) in THF (25 mL) was added dropwise a solution of 1e (1.00 g, 2.31 mmol) in THF (20 mL). The reaction was stirred at rt for 6 h under argon and then quenched with water and acidified to pH ˜1 by carefully adding conc. HCl. The mixture was extracted with CH₂Cl₂ (3×50 mL). The combined organic layers were washed subsequently with brine (1×50 mL) and water (1×50 mL), dried over MgSO₄, and evaporated to dryness to give R1ArMT (0.83 g, 0.20 mmol, 88%). ¹H NMR (500 MHZ, CDCl₃): δ 0.87 (t, J=6.8 Hz, ArOCH₂CH₂(CH₂)₁₅CH₃, 3H), 1.21-1.50 (m, ArOCH₂CH₂(CH₂)₁₅CH₃, and ArCH₂CH₂SH, 31H), 1.75 (pent, J=6.8 Hz, ArOCH₂CH₂(CH₂)₁₅CH₃, 2H), 2.74 (m, ArCH₂CH₂SH), 2.83 (t, J=7.4 Hz, ArCH₂CH₂SH, 2H), 3.92 (t, J=6.9 Hz, ArOCH₂CH₂(CH₂)₁₅CH₃, 2H), 6.83 (d, J=8.6 Hz, ArH, 2H), 7.09 (d, J=8.6 Hz, ArH, 2H). ¹³CNMR (125 MHZ, CDCl₃): δ 14.22, 22.78, 26.14, 26.44, 29.45, 29.78, 32.01, 39.46, 68.10, 114.56, 129.66, 131.76, 157.93.

### 2-(3,5-Bis(octadecyloxy)phenyl)ethanol (2f)

Following the procedure described for 1f, a solution of 2a (2.00 g, 2.91 mmol) in THF (15 mL) was added to a suspension of LiAlH₄ (0.28 g, 7.2 mmol) in THF (10 mL) to obtain 2f (1.70 g, 2.58 mmol, 89%). ¹H NMR (300 MHZ, CDCl₃): δ 0.88 (t, J=6.3 Hz, Ar(OCH₂CH₂(CH₂)₁₅CH₃)₂, 6H), 1.24-1.43 (m, Ar(OCH₂CH₂(CH₂)₁₅CH₃)₂, 60H), 1.76 (pent, J=6.3 Hz, Ar(OCH₂CH₂(CH₂)₁₅CH₃)₂, 4H), 2.79 (t, J=6.3 Hz ArCH₂CH₂OH, 2H), 3.83-3.890 (m, ArCH₂CH₂OH, and ArCH₃CH₂OH, 3H), 3.91 (t, J=6.6 Hz, Ar(OCH₂CH₂(CH₂)₁₅CH₃)₂, 4H), 6.35 (s, ArH, 1H), 6.36 (s, ArH, 2H).

### 3,5-Bis(octadecyloxy)phenethylmethanesulfonate (2g)

Following the procedure described for 1g, a mixture of 2f (2.00 g, 3.03 mmol) and triethylamine (1.27 mL, 9.10 mmol) in THF (30 mL) was treated with methansulfonyl chloride (0.71 mL, 9.1 mmol) to obtain the crude mesylate 2g (1.94 g, 2.65 mmol, 87%) as a white powder. ¹H NMR (300 MHZ, CDCl₃): δ 0.88 (t, J=6.3 Hz, Ar(OCH₂CH₂(CH₂)₁₅CH₃)₂, 6H), 1.20-1.42 (m, Ar(OCH₂CH₂(CH₂)₁₅CH₃)₂, 60H), 1.76 (pent, J=6.6 Hz, Ar(OCH₂CH₂(CH₂)₁₅CH₃)₂, 4H), 2.88 (s, ArCH₂CH₂SO₂CH₃, 3H), 2.97 (t, J=6.6 Hz, ArCH₂CH₂SO₂CH₃, 2H), 3.91 (t, J=6.6 Hz, ArOCH₂CH₂(CH₂)₁₅CH₃, 2H), 4.40 (t, J=6.6 Hz, ArCH₂CH₂SO₂CH₃, 2H), 6.63 (s (br), ArH, 3H).

### 1,3-Bis(octadecyloxy)-5-(2-thiocyanatoethyl)benzene (2h)

Following the procedure described for 1 h, 2g (2.00 g, 2.73 mmol) was treated with KSCN (2.66 g, 27.3 mmol) dissolved in a mixture of EtOH (10 mL) and DMF (10 mL). The crude product was purified by column chromatography using CH₂Cl₂:hexane (3:2) as the eluent to afford 2e (1.52 g, 2.17 mmol, 79%). ¹H NMR (500 MHZ, CDCl₃): δ 0.88 (t, J=6.9 Hz, Ar(OCH₂CH₂(CH₂)₁₅CH₃)₂, 6H), 1.21-1.49 (m, Ar(OCH₂CH₂(CH₂)₁₅CH₃)₂, 60H), 1.77 (pent, J=6.9 Hz, Ar(OCH₂CH₂(CH₂)₁₅CH₃)₂, 4H), 3.02 (t, J=6.9 Hz, ArCH₂CH₂SCN, 2H), 3.16 (t, J=6.8 Hz, ArCH₂CH₂SCN, 2H), 3.92 (t, J=6.9 Hz, Ar(OCH₂CH₂(CH₂)₁₅CH₃)₂, 4H), 6.32 (s, ArH, 2H), 6.34 (s, ArH, 1H).

### 2-(3,3-Bis(octadecyloxy)phenyl)ethanethiol (R2ArMT)

Following the procedure described for R1ArMT, a solution of 2h (2.00 g, 2.91 mmol) in THF (15 mL) was added dropwise to a suspension of LiAlH₄ (0.28 g, 7.2 mmol) in THF (10 mL) to give R2ArMT (0.75 g, 1.1 mmol, 78%). ¹H NMR (500 MHZ, CDCl₃): δ 0.87 (t, J=6.9 Hz, Ar(OCH₂CH₂(CH₂)₁₅CH₃)₂, 6H), 1.21-1.50 (m, Ar(OCH₂CH₂(CH₂)₁₅CH₃)₂, and ArCH₂CH₂SH, 61H), 1.75 (pent, J=6.9 Hz, Ar(OCH₂CH₂(CH₂)₁₅CH₃)₂, 4H), 2.76 (m, ArCH₂CH₂SH, 2H), 2.83 (t, J=6.9 Hz, ArCH₂CH₂SH, 2H), 3.90 (t, J=6.9 Hz, Ar(OCH₂CH₂(CH₂)₁₅CH₃)₂, 4H), 6.32 (s, ArH, 3H). ¹³CNMR (125 MHZ, CDCl₃): d 14.22, 22.79, 25.94, 26.15, 29.59, 29.70, 29.78, 32.02, 40.67, 68.08, 99.34, 107.22, 142.01, 160.46.

### 2-(3,4,5-Tris(octadecyloxy)phenyl)ethanol (31)

Following the procedure described for 1f, a solution of 3g (2.00 g, 2.09 mmol) in THF (15 mL) was added dropwise to a suspension of LiAlH₄ (0.20 g, 5.2 mmol) in THF (10 mL) to give 3h (1.70 g, 1.83 mmol, 88%). ¹H NMR (400 MHZ, CDCl₃): δ 0.87 (t, J=7.4 Hz, Ar(OCH₂CH₂(CH₂)₁₅CH₃)₃, 9H), 1.25-1.45 (m, Ar(OCH₂CH₂(CH₂)₁₅CH₃)₃, 90H), 1.65-1.87 (m, Ar(OCH₂CH₂(CH₂)₁₅CH₃)₃, 6H), 2.76 (t, J=8.0 Hz, ArCH₂CH₂OH, 2H), 3.85 (m, ArCH₂CH₂OH, 1H), 3.92-3.96 (m, Ar(OCH₂CH₂(CH₂)₁₅CH₃)₃ and ArCH₂CH₂OH, 8H), 6.37 (s, ArH, 2H).

### 3,4,5-Tris(octadecyloxy)phenylmethanesulfonate (3m)

Following the procedure described for 1g, a mixture of 3l (1.50 g, 1.62 mmol) and triethylamine (0.68 mL, 4.8 mmol) in THF (30 mL) was treated with methanesulfonyl chloride (0.38 mL, 4.8 mmol) to obtain the crude mesylate 3l. The crude product was dissolved in a minimum volume of CH₂C21, and MeOH was added to precipitate 31 (1.38 g, 1.37 mmol, 85%) as a white powder. ¹H NMR (500 MHZ, CDCl₃): δ 0.87 (t, J=7.4 Hz, Ar(OCH₂CH₂(CH₂)₁₅CH₃)₃, 9H), 1.20-1.48 (m, Ar(OCH₂CH₂(CH₂)₁₅CH₃)₃, 90H), 1.69-1.80 (m, Ar(OCH₂CH₂(CH₂)₁₅CH₃)₃, 6H), 2.85 (s, ArCH₂CH₂SO₂CH₃, 3H), 2.95 (t, J=7.4 Hz, ArCH₂CH₂SO₂CH₃, 2H), 3.90-3.96 (m, Ar(OCH₂CH₂(CH₂)₁₅CH₃)₃, 6H), 4.38 (t, J=7.4 Hz, ArCH₂CH₂SO₂CH₃, 2H), 6.37 (s, ArH, 2H).

### 1,2,3-Tris(octadecyloxy(-5-(2-thiocyanatoethyl)benzene (3n)

Following the procedure described for 1h, 3m (2.00 g, 1.99 mmol) was treated with KSCN (1.93 g, 19.9 mmol) dissolved in a mixture of EtOH (10 mL) and DMF (10 mL). The crude product was purified by column chromatography using CH₂Cl₂:hexane (3:2) as the eluent to afford 3n (1.50 g, 1.54 mmol, 78%). ¹H NMR (400 MHZ, CDCl₃): δ 0.87 (t, J=6.9 Hz, Ar(OCH₂CH₂(CH₂)₁₅CH₃)₃, 9H), 1.24-1.49 (m, Ar(OCH₂CH₂(CH₂)₁₅CH₃)₃, 90H), 1.75-1.81 (m, Ar(OCH₂CH₂(CH₂)₁₅CH₃)₃, 6H), 3.06 (t, J=7.4 Hz, ArCH₂CH₂SCN, 2H), 3.14 (t, J=7.4 Hz, ArCH₂CH₂SCN, 2H), 3.92 (m, Ar(OCH₂CH₂(CH₂)₁₅CH₃)₃, 6H), 6.37 (s, ArH, 2H).

### 2-(3,4,5-Tris(octadecyloxy)phenyl)ethanethiol (R3ArMT)

Following the procedure described for R1ArMT, a solution of 3n (1.00 g, 1.03 mmol) in THF (15 mL) was added dropwise to a suspension of LiAlH₄ (0.10 g, 2.6 mmol) in THF (10 mL) to give the crude product. Purification was performed by column chromatography with CH₂Cl₂:hexane (3:2) as the eluent to afford pure R3ArMT (0.67 g, 0.71 mmol, 69%) as a white powder. ¹H NMR (500 MHZ, CDCl₃): δ 0.87 (t, J=6.9 Hz, Ar(OCH₂CH₂(CH₂)₁₅CH₃)₃, 9H), 1.25-1.49 (m, Ar(OCH₂CH₂(CH₂)₁₅CH₃)₃ and ArCH₂CH₂SH, 91H), 1.75-1.81 (m, Ar(OCH₂CH₂(CH₂)₁₅CH₃)₃, 6H), 2.76 (m, ArCH₂CH₂SH, 2H), 2.80 (t, J=6.9 Hz, ArCH₂CH₂SCN, 2H), 3.90 (m, Ar(OCH₂CH₂(CH₂)₁₅CH₃)₃, 6H), 6.36 (s, ArH, 2H). ¹³CNMR (500 MHZ, CDCl₃): 14.22, 22.79, 26.21, 26.24, 29.47, 29.54, 29.75, 29.81, 30.43, 32.02, 40.70, 62.23, 73.51, 107.25, 135.00, 136.92, 153.17.

### Preparation of SAMs

Materials and Methods

Gold shot (99.99%) was purchased from Americana Precious Metals. Chromium rods (99.9%) were purchased from R. D. Mathis Company. Polished single-crystal Si(100) wafers were purchased from Silicon Inc. and rinsed with absolute ethanol (Aaper Alcohol and Chemical Co.) before use. The contacting liquids used for wettability measurements were the highest purity available from Aldrich Chemical Co. and were used without further purification. The starting material, and chemicals used in the syntheses of all adsorbates were mostly purchased from Aldrich Chemical Co. Solvents used in the syntheses were distilled over calcium hydride and stored under argon. Column chromatography was performed using silica gel (40-64 mm) and thin-layer chromatography (TLC) was carried out using 200 mm-thick silica gel plates, which were purchased from Sorbent Technologies. Inc. Nuclear Magnetic Resonance (NMR) spectra were recorded on JOEL ECX-400 and ECA-500 spectrometers operating at 400 MHZ and 500 MHZ, respectively. The data were obtained in CDCl₃ and referenced to δ 7.26 and 77.00 ppm for ¹H NMR and ¹³C NMR, respectively.

Preparation of SAMs

Under a vacuum, ˜10 Torr, a thin layer (100 Å) of chromium was first evaporated onto polished silicon (100) wafers to assist the adhesion of gold on the silicon substrate. Subsequently, the deposition of 1000 Å of gold onto the chromium-coated silicon wafers was performed. The resultant wafers were rinsed with absolute ethanol and blown dry with ultra-pure nitrogen before use. The freshly prepared gold-coated wafers were cut into slides (1×4 cm) and the slides were cleaned by rinsing with absolute ethanol and blown dry with ultra-pure nitrogen before collecting the optical constants of the substrates by ellipsometry. The glass vials used to store each thiol solution were previously cleaned with piranha solution (3:1 mixture of H₂SO₄/H₂O₂) and rinsed thoroughly with deionized water, followed by absolute ethanol. [Caution: Piranha solution is highly corrosive, should never be stored, and should be handled with extreme care.] The slides were then immersed in the appropriate 1 mM thiol solution and allowed to equilibrate for 72 hours at room temperature. Samples were rinsed with toluene, THF, methanol, and then ethanol, successively, and dried with a flow of ultra-pure nitrogen before characterization.

Characterization of SAMs

Ellipsometric Measurements

A Rudolph Research Auto EL III ellipsometer equipped with a He—Ne laser (632.8 nm) at angle of incidence of 70° was utilized to measure the film thicknesses. An approximate value of film refractive index of 1.45 was applied for all measurements. For each sample, optical constants were determined from six measurements from two separate slides using three different spots for each slide. Reported values of thicknesses were the average of the measurements over the experimental collections.

Contact Angle Measurements

Contact angles for the SAMs were measured on a Ramé-Hart model 100 contact angle goniometer. The contacting liquid water (H₂O), hexadecane (HD), and decalin (DEC) were dispensed (advancing angle, qₐ) and withdrawn (receding angle, qᵣ) on the surfaces using a Matrix Technologies micro-Electrapette 25 at the slowest speed (1 mL/s). The contact angles were carried out at room temperature while keeping the pipet tip in contact with the drop. Contact angle values were reported by averaging the results from six independent drops from two separate slides with three drops per slide, including measurements for both drop edges.

X-ray Photoelectron Spectroscopy (XPS)

XPS spectra were recorded with a PHI 5700×-ray photoelectron spectrometer equipped with a monochromatic Al Ka X-ray source (hn=1486.7 eV) incident at 900 relative to the axis of the hemispherical energy analyzer. Spectra were taken at high resolution with a pass energy of 23.5 eV, a photoelectron taken off angle of 45° from the surface, and an analyzer spot diameter of 2.0 mm. The pressure in the chamber during the measurements was ˜4×10⁻⁸ Torr. The binding energies were referenced to the Au 4f₇₁₂ peak at 84.0 eV.

Polarization Modulation Infrared Reflection Absorption Spectroscopy (PM-IRRAS)

IR spectra were collected with a Nicolet NEXUS 670 Fourier transform IR spectrometer equipped with a liquid-nitrogen-cooled mercury-cadmium-telluride (MCT) detector and a Hinds Instrument PEM-90 photoelastic modulator. All measurements were performed in a sample compartment purged with nitrogen gas during the course of the experiments. The spectra were collected at 2 cm⁻¹ spectral resolution for 512 scans with a grazing angle for the infrared beam aligned at 80°.

Thermal Desorption of SAMs at the Elevated Temperature

Thermal stability experiments were performed in isooctane at 80±2° C. SAM-coated gold slides were immersed into the stirred solvent and periodically removed at systematic intervals of time. The samples were subsequently rinsed with toluene, THF, methanol, and ethanol, in that order, and blown dry in a stream of ultra-pure nitrogen. The samples were immediately characterized by ellipsometry and then reimmersed in the heating solvent.

While the invention described herein specifically focuses on methods for the preparation of stable homogeneously mixed thin film coatings through the use of tailored aromatic-based adsorbates, one of ordinary skills in the art, with the benefit of this disclosure, would recognize the extension of such approach to other systems.

### REFERENCES OF THE INVENTION

The following references were cited in the specification:
1. Gates, B. D.; Xu, Q.; Stewart, M.; Ryan, D.; Willson, C. G.; Whitesides, G. M. Chem. Rev. (Washington, D.C., U.S.) 2005, 105, 1171.
2. DiBenedetto, S. A.; Facchetti, A.; Ratner, M. A.; Marks, T. J. Adv. Mater. (Weinheim, Ger.) 2009, 21, 1407.
3. Katsonis, N.; Lubomska, M.; Pollard, M. M.; Fering a, B. L.; Rudolf, P. Prog. Surf. Sci. 2007, 82, 407.
4. Carpick, R. W.; Salmeron, M. Chem. Rev. 1997, 97, 1163.
5. Nakano, M.; Ishida, T.; Sano, H.; Sugimura, H.; Miyake, K.; Ando, Y.; Sasaki, S. Appl. Surf. Sci. 2008, 255, 3040.
6. Sinapi, F.; Lejeune, I.; Delhalle, J.; Mekhalif, Z. Electrochim. Acta 2007, 52, 5182.
7. Zamborini, F. P.; Campbell, J. K.; Crooks, R. M. Langmuir 1998, 14, 640.
8. Bhushan, B.; Kasai, T.; Kulik, G.; Barbieri, L.; Hoffmann, P. Ultramicroscopy 2005, 105, 176.
9. Liu, H.; Bhushan, B. Ultramicroscopy 2002, 91, 185.
10. Frasconi, M.; Mazzei, F.; Ferri, T. Anal. Bioanal. Chem. 2010, 398, 1545.
11. Chaki, N. K.; Vijayamohanan, K. Biosens. Bioelectron. 2002, 17, 1.
12. Wyszogrodzka, M.; Haag, R. Biomacromolecules 2009, 10, 1043.
13. Wyszogrodzka, M.; Haag, R. Langmuir 2009, 25, 5703.
14. Troughton, E. B.; Bain, C. D.; Whitesides, G. M.; Nuzzo, R. G.; Allara, D. L.; Porter, M. D. Langmuir 1988, 4, 365.
15. Nuzzo, R. G.; Allara, D. L. J. Am. Chem. Soc. 1983, 105, 4481.
16. Chuang, W.-H.; Lin, J.-C. J. Biomed. Mater. Res., Part A 2007, 82A, 820.
17. Ingram, R. S.; Hostetler, M. J.; Murray, R. W. J. Am. Chem. Soc. 1997, 119, 9175.
18. Shon, Y.-S.; Colorado, R., Jr.; Williams, C. T.; Bain, C. D.; Lee, T. R. Langmuir 2000, 16, 541.
19. Shon, Y.-S.; Lee, S.; Colorado, R., Jr.; Perry, S. S.; Lee, T. R. J. Am. Chem. Soc. 2000, 122, 7556.
20. Shon, Y.-S.; Lee, T. R. J. Phys. Chem. B 2000, 104, 8192.
21. Garg, N.; Carrasquillo-Molina, E.; Lee, T. R. Langmuir 2002, 18, 2717.
22. Evans, S. D.; Urankar, E.; Ulman, A.; Ferris, N. J. Am. Chem. Soc. 1991, 113, 4121.
23. Molecular models were performed using the Gaussview03, revision 3.07; Gaussian, Inc., Pittsburgh, Pa. We assumed that the alkyl chains were fully extended and tilted ˜30 from the surface normal. The following data were used: C-C˜1.5 A, C-O˜1.4 A, <CCC˜109.5 and <COC˜110.8
24. Shnidman, Y.; Ulman, A.; Eilers, J. E. Langmuir 1993, 9, 1071.
25. Shon, Y.-S.; Lee, T. R. Langmuir 1999, 15, 1136.
26. Castner, D. G.; Hinds, K.; Grainger, D. W. Langmuir 1996, 12, 5083.
27. Ishida, T.; Choi, N.; Mizutani, W.; Tokumoto, H.; Kojima, I.; Azehara, H.; Hokari, H.; Akiba, U.; Fujihira, M. Langmuir 1999, 15, 6799.
28. Heeg, J.; Schubert, U.; Kuchenmeister, F. Fresenius' J. Anal. Chem. 1999, 365, 272.
29. To compare the packing density relative to the densely packed C18 SAM as a reference, the integration of S 2p spectra of the SAMs derived from bi-, and tridentrate adsorbates were divided by factors of two, and three, respectively.
30. Bethencourt, M. I.; Srisombat, L.-o.; Chinwangso, P.; Lee, T. R. Langmuir 2009, 25, 1265.
31. Barriet, D.; Yam, C. M.; Shmakova, O. E.; Jamison, A. C.; Lee, T. R. Langmuir 2007, 23, 8866.
32. Lim, J. K.; Kim, Y.; Kwon, O.; Joo, S.-W. ChemPhysChem 2008, 9, 1781.
33. Murty, K. V. G. K.; Venkataramanan, M.; Pradeep, T. Langmuir 1998, 14, 5446.
34. Tillman, N.; Ulman, A.; Schildkraut, J. S.; Penner, T. L. J. Am. Chem. Soc. 1988, 110, 6136.
35. Ulman, A.; Scaringe, R. P. Langmuir 1992, 8, 894.
36. Chechik, V.; Schoenherr, H.; Vancso, G. J.; Stirling, C. J. M. Langmuir 1998, 14, 3003.
37. Lenk, T. J.; Hallmark, V. M.; Hoffmann, C. L.; Rabolt, J. F.; Castner, D. G.; Erdelen, C.; Ringsdorf, H. Langmuir 1994, 10, 4610.
38. Porter, M. D.; Bright, T. B.; Allara, D. L.; Chidsey, C. E. D. J. Am. Chem. Soc. 1987, 109, 3559.
39. Snyder, R. G.; Strauss, H. L.; Elliger, C. A. J. Phys. Chem. 1982, 86, 5145.
40. MacPhail, R. A.; Strauss, H. L.; Snyder, R. G.; Elliger, C. A. J. Phys. Chem. 1984, 88, 334.
41. Bensebaa, F.; Ellis, T. H.; Badia, A.; Lennox, R. B. Langmuir 1998, 14, 2361.
42. Tao, Y. T.; Lee, M. T.; Chang, S. C. J. Am. Chem. Soc. 1993, 115, 9547.
43. Chang, S.-C.; Chao, I.; Tao, Y.-T. J. Am. Chem. Soc. 1994, 116, 6792.
44. Valiokas, R.; Oestblom, M.; Svedhem, S.; Svensson, S. C. T.; Liedberg, B. J. Phys. Chem. B 2002, 106, 10401.
45. Bensebaa, F.; Ellis, T. H.; Badia, A.; Lennox, R. B. J. Vac. Sci. Technol., A 1995, 13, 1331.
46. Blaudez, D.; Buffeteau, T.; Cornut, J. C.; Desbat, B.; Escafre, N.; Pezolet, M.; Turlet, J. M. Thin Solid Films 1994, 242, 146.
47. Mendelsohn, R.; Brauner, J. W.; Gericke, A. Annu. Rev. Phys. Chem. 1995, 46, 305.
48. Snyder, R. G.; Scherer, J. R. J. Chem. Phys. 1979, 71, 3221.
49. Snyder, R. G.; Hsu, S. L.; Krimm, S. Spectrochim. Acta, Part A 1978, 34A, 395.
50. Hill, I. R.; Levin, I. W. J. Chem. Phys. 1979, 70, 842.
51. Laibinis, P. E.; Whitesides, G. M.; Allara, D. L.; Tao, Y. T.; Parikh, A. N.; Nuzzo, R. G. J. Am. Chem. Soc. 1991, 113, 7152.
52. Nuzzo, R. G.; Dubois, L. H.; Allara, D. L. J. Am. Chem. Soc. 1990, 112, 558.
53. Porter, M. D.; Bright, T. B.; Allara, D. L.; Chidsey, C. E. D. J. Am. Chem. Soc. 1987, 109, 3559.
54. Bain, C. D.; Troughton, E. B.; Tao, Y. T.; Evall, J.; Whitesides, G. M.; Nuzzo, R. G. J. Am. Chem. Soc. 1989, 111, 321.
55. Parikh, A. N.; Allara, D. L. J. Chem. Phys. 1992, 96, 927.
56. Wood, K. A.; Snyder, R. G.; Strauss, H. L. J. Chem. Phys. 1989, 91, 5255.
57. Zerbi, G.; Roncone, P.; Longhi, G.; Wunder, S. L. J. Chem. Phys. 1988, 89, 166.
58. Whitesides, G. M.; Laibinis, P. E. Langmuir 1990, 6, 87.
59. Bain, C. D.; Whitesides, G. M. Angew. Chem. 1989, 101, 522.
60. Timmons, C. O.; Zisman, W. A. J. Colloid Interface Sci. 1966, 22, 165.
61. Lee, S.; Park, J.-S.; Lee, T. R. Langmuir 2008, 24, 4817.
62. Park, J.-S.; Vo, A. N.; Barriet, D.; Shon, Y.-S.; Lee, T. R. Langmuir 2005, 21, 2902.
63. Wenzl, I.; Yam, C. M.; Barriet, D.; Lee, T. R. Langmuir 2003, 19, 10217.
64. Strong, L.; Whitesides, G. M. Langmuir 1988, 4, 546.
65. Chidsey, C. E. D.; Liu, G. Y.; Rowntree, P.; Scoles, G. J. Chem. Phys. 1989, 91, 4421.
66. Walczak, M. M.; Alves, C. A.; Lamp, B. D.; Porter, M. D. J. Electroanal. Chem. 1995, 396, 103.
67. Purcell, K. F.; Kotz, J. C., Inorganic Chemistry. W. B. Saunders: Philadelphia, 1977.
68. Jennings, G. K.; Laibinis, P. E. J. Am. Chem. Soc. 1997, 119, 5208.
69. Jennings, G. K.; Laibinis, P. E. Langmuir 1996, 12, 6173.

All references cited herein are incorporated by reference. Although the invention has been disclosed with reference to its preferred embodiments, from reading this description those of skill in the art may appreciate changes and modification that may be made which do not depart from the scope and spirit of the invention as described above and claimed hereafter.

We claim:

1. A method for preparing homogeneously self-assembled monolayers (SAMs) comprising:
adsorbing a mono-dentate absorbate, a bi-dentate absorbate, a tri-dentate absorbate or a mixture or combination thereof onto a surface of a substrate to form a SAM modified surface,
where the adsorbates comprise an aromatic ring including one dentate head group or a plurality of dentate head groups and one tunable tail group or a plurality of tunable tail groups, and
where the SAM exhibit: (1) homogenous lateral chain distributions, (2) no or substantially no phase separation or islanding across SAM modified surfaces, and (3) enhanced stability as compared to a corresponding linear alkyl thiol monodentate adsorbate.

2. The method of claim 1, wherein the surface comprises a metal surface.

3. The method of claim 1, wherein the absorbate or mixture thereof comprise one monodentate adsorbate or a mixture of monodentate absorbates.

4. The method of claim 1, wherein the absorbate or mixture thereof comprise one bidentate adsorbate or a mixture of bidentate absorbates.

5. The method of claim 4, wherein the bidentate adsorbate or the mixture of bidentate absorbates comprise a non-symmetrical spiroalkanedithiol or a mixture of non-symmetrical spiroalkanedithiols.

6. The method of claim 1, wherein the absorbate or mixture thereof comprise one tridentate adsorbate or a mixture of tridentate absorbates.

6. The method of claim 1, wherein the absorbate or mixture thereof comprise compounds of the general formulas (I-V):
Z¹,Z²,Z³-A(SH)ₖ(RSH)ₗ(CHₘ₁(SH)ₘ₂)ₙ (I)
where: (1) A is an aromatic group, where one of the carbon atoms of the group may be replaced by an oxygen atom (O), a nitrogen atom (N), or a sulfur atom (S), (2) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹, Z², or Z³ groups is not a hydrogen atom, (3) R is the same or different carbyl groups having between 1 and 3 carbon atoms, (4) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (5) the R^{a2-a4} may be linear or branched carbyl groups having between about 12 to 40 carbon atoms, (6) the R^{d,f,x} groups may be hydrogen atoms or linear or branched carbyl groups having between 1 and 10 carbon atoms, (7) R^{g,h,i,w,z} are linear or branched carbyl groups having between 1 and 10 carbon atoms, (8) k, l, m1, m2 and n are integers, (9) k has a value between 1 and 3, when l and n have a value of 0, (10) l has a value between 1 and 3, when k and n have a value of 0, (11) n has a value of 1, when k and l have a value of 0, and (12) m1+m2 is equal to 3. In certain embodiments, at least two of Z¹, Z², and Z³ groups are not hydrogen atoms. In other embodiments, all of the Z¹, Z², and Z³ groups are not hydrogen atoms;
Z¹,Z²,Z³-ACHₘ₁(SH)ₘ₂ (II)
where: (1) A is an aromatic group, where one of the carbon atoms of the group may be replaced by an oxygen atom (O), a nitrogen atom (N), or a sulfur atom (S), (2) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹, Z², or Z³ groups is not a hydrogen atom, (3) R is the same or different carbyl groups having between 1 and 3 carbon atoms, (4) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (5) the R^{a2-a4} may be linear or branched carbyl groups having between about 12 to 40 carbon atoms, (6) the R^{d,f,x} groups may be hydrogen atoms or linear or branched carbyl groups having between 1 and 10 carbon atoms, (7) R^{g,h,i,w,z} are linear or branched carbyl groups having between 1 and 10 carbon atoms, (8) m1 and m2 are integers, and (9) m1+m2 is equal to 3;
Z¹,Z²,Z³-A-m-(RSH)₂ (III)
where: (1) A is an aromatic group, where one of the carbon atoms of the group may be replaced by an oxygen atom (O), a nitrogen atom (N), or a sulfur atom (S), (2) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹, Z², or Z³ groups is not a hydrogen atom, (3) R is the same or different carbyl groups having between 1 and 3 carbon atoms, (4) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (5) the R^{a2-a4} may be linear or branched carbyl groups having between about 12 to 40 carbon atoms, (6) the R^{d,f,x} groups may be hydrogen atoms or linear or branched carbyl groups having between 1 and 10 carbon atoms, and (7) R^{g,h,i,w,z} are linear or branched carbyl groups having between 1 and 10 carbon atoms;
Z¹,Z²,Z³-Py-m-(RSH)₂ (IV)
where: (1) Py is pyridine, (2) -m- means the two RSH groups meta to each other, (3) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹, Z², and Z³ groups is not hydrogen, (4) R is the same or different carbyl groups having between 1 and 3 carbon atoms, (5) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (6) the R^{a2-a4} may be linear or branched carbyl groups having between about 12 to 40 carbon atoms, (7) the R^{d,f,x} groups may be hydrogen atoms or linear or branched carbyl groups having between 1 and 10 carbon atoms, and (8) R^{g,h,i,w,z} are linear or branched carbyl groups having between 1 and 10 carbon atoms; and
Z¹,Z²-Cp-m-(RSH)₂ (V)
where: (1) Cp is 5-member aromatic ring, where one of the carbon atoms of the Cp ring may be replaced by an oxygen atom, a nitrogen atom, or a sulfur atom, (2) -m- means that the two RSH groups are meta to each other or occupy the 2 and 5 positions of the five membered Cp ring, (3) Z¹ and Z² groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹ and Z² groups is not a hydrogen atom, (4) R is the same or different carbyl groups having between 1 and 3 carbon atoms, (5) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (6) the R^{a2-a4} may be linear or branched carbyl groups having between about 12 to 40 carbon atoms, (7) the R^{d,f,x} groups may be hydrogen atoms or linear or branched carbyl groups having between 1 and 10 carbon atoms, and (8) R^{g,h,i,w,z} are linear or branched carbyl groups having between 1 and 10 carbon atoms.

7. The method of claim 1, wherein the absorbate or mixture thereof comprise compounds of the general formula (I):
Z¹,Z²,Z³-A(SH)ₖ(RSH)ₗ(CHₘ₁(SH)ₘ₂)ₙ (I)
where: (1) A is an aromatic group, where one of the carbon atoms of the group may be replaced by an oxygen atom (O), a nitrogen atom (N), or a sulfur atom (S), (2) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹, Z², or Z³ groups is not a hydrogen atom, (3) R is the same or different carbyl groups having between 1 and 3 carbon atoms, (4) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (5) the R^{a2-a4} may be linear or branched carbyl groups having between about 12 to 40 carbon atoms, (6) the R^{d,f,x} groups may be hydrogen atoms or linear or branched carbyl groups having between 1 and 10 carbon atoms, (7) R^{g,h,i,w,z} are linear or branched carbyl groups having between 1 and 10 carbon atoms, (8) k, l, m1, m2 and n are integers, (9) k has a value between 1 and 3, when l and n have a value of 0, (10) l has a value between 1 and 3, when k and n have a value of 0, (11) n has a value of 1, when k and l have a value of 0, and (12) m1+m2 is equal to 3. In certain embodiments, at least two of Z¹, Z², and Z³ groups are not hydrogen atoms. In other embodiments, all of the Z¹, Z², and Z³ groups are not hydrogen atoms.

8. The method of claim 1, wherein the absorbate or mixture thereof comprise compounds of the general formula (II):
Z¹,Z²,Z³-ACHₘ₁(SH)ₘ₂ (II)
where: (1) A is an aromatic group, where one of the carbon atoms of the group may be replaced by an oxygen atom (O), a nitrogen atom (N), or a sulfur atom (S), (2) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹, Z², or Z³ groups is not a hydrogen atom, (3) R is the same or different carbyl groups having between 1 and 3 carbon atoms, (4) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (5) the R^{a2-a4} may be linear or branched carbyl groups having between about 12 to 40 carbon atoms, (6) the R^{d,f,x} groups may be hydrogen atoms or linear or branched carbyl groups having between 1 and 10 carbon atoms, (7) R^{g,h,i,w,z} are linear or branched carbyl groups having between 1 and 10 carbon atoms, (8) m1 and m2 are integers, and (9) m1+m2 is equal to 3.

9. The method of claim 1, wherein the absorbate or mixture thereof comprise compounds of the general formula (III):
Z¹,Z²,Z³-A-m-(RSH)₂ (III)
where: (1) A is an aromatic group, where one of the carbon atoms of the group may be replaced by an oxygen atom (O), a nitrogen atom (N), or a sulfur atom (S), (2) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹, Z², or Z³ groups is not a hydrogen atom, (3) R is the same or different carbyl groups having between 1 and 3 carbon atoms, (4) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (5) the R^{a2-a4} may be linear or branched carbyl groups having between about 12 to 40 carbon atoms, (6) the R^{d,f,x} groups may be hydrogen atoms or linear or branched carbyl groups having between 1 and 10 carbon atoms, and (7) R^{g,h,i,w,z} are linear or branched carbyl groups having between 1 and 10 carbon atoms.

10. The method of claim 1, wherein the absorbate or mixture thereof comprise compounds of the general formula (IV):
Z¹,Z²,Z³-Py-m-(RSH)₂ (IV)
where: (1) Py is pyridine, (2) -m- means the two RSH groups meta to each other, (3) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹, Z², and Z³ groups is not hydrogen, (4) R is the same or different carbyl groups having between 1 and 3 carbon atoms, (5) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (6) the R^{a2-a4} may be linear or branched carbyl groups having between about 12 to 40 carbon atoms, (7) the R^{d,f,x} groups may be hydrogen atoms or linear or branched carbyl groups having between 1 and 10 carbon atoms, and (8) R^{g,h,i,w,z} are linear or branched carbyl groups having between 1 and 10 carbon atoms.

11. The method of claim 1, wherein the absorbate or mixture thereof comprise compounds of the general formula (V):
Z¹,Z²-Cp-m-(RSH)₂ (V)
where: (1) Cp is 5-member aromatic ring, where one of the carbon atoms of the Cp ring may be replaced by an oxygen atom, a nitrogen atom, or a sulfur atom, (2) -m- means that the two RSH groups are meta to each other or occupy the 2 and 5 positions of the five membered Cp ring, (3) Z¹ and Z² groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹ and Z² groups is not a hydrogen atom, (4) R is the same or different carbyl groups having between 1 and 3 carbon atoms, (5) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (6) the R^{a2-a4} may be linear or branched carbyl groups having between about 12 to 40 carbon atoms, (7) the R^{d,f,x} groups may be hydrogen atoms or linear or branched carbyl groups having between 1 and 10 carbon atoms, and (8) R^{g,h,i,w,z} are linear or branched carbyl groups having between 1 and 10 carbon atoms.

12. A self-assembled monolayer composition comprising an absorbate or mixture thereof comprise compounds of the general formulas (I-V):
Z¹,Z²,Z³-A(SH)ₖ(RSH)ₗ(CHₘ₁(SH)ₘ₂)ₙ (I)
where: (1) A is an aromatic group, where one of the carbon atoms of the group may be replaced by an oxygen atom (O), a nitrogen atom (N), or a sulfur atom (S), (2) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR_{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹, Z², or Z³ groups is not a hydrogen atom, (3) R is the same or different carbyl groups having between 1 and 3 carbon atoms, (4) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (5) the R^{a2-a4} may be linear or branched carbyl groups having between about 12 to 40 carbon atoms, (6) the R^{d,f,x} groups may be hydrogen atoms or linear or branched carbyl groups having between 1 and 10 carbon atoms, (7) R^{g,h,i,w,z} are linear or branched carbyl groups having between 1 and 10 carbon atoms, (8) k, l, m1, m2 and n are integers, (9) k has a value between 1 and 3, when l and n have a value of 0, (10) l has a value between 1 and 3, when k and n have a value of 0, (11) n has a value of 1, when k and l have a value of 0, and (12) m1+m2 is equal to 3. In certain embodiments, at least two of Z¹, Z², and Z³ groups are not hydrogen atoms. In other embodiments, all of the Z¹, Z², and Z³ groups are not hydrogen atoms;
Z¹,Z²,Z³-ACHₘ₁(SH)ₘ₂ (II)
where: (1) A is an aromatic group, where one of the carbon atoms of the group may be replaced by an oxygen atom (O), a nitrogen atom (N), or a sulfur atom (S), (2) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹, Z², or Z³ groups is not a hydrogen atom, (3) R is the same or different carbyl groups having between 1 and 3 carbon atoms, (4) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (5) the R^{a2-a4} may be linear or branched carbyl groups having between about 12 to 40 carbon atoms, (6) the R^{d,f,x} groups may be hydrogen atoms or linear or branched carbyl groups having between 1 and 10 carbon atoms, (7) R^{g,h,i,w,z} are linear or branched carbyl groups having between 1 and 10 carbon atoms, (8) m1 and m2 are integers, and (9) m1+m2 is equal to 3;
Z¹,Z²,Z³-A-m-(RSH)₂ (III)
where: (1) A is an aromatic group, where one of the carbon atoms of the group may be replaced by an oxygen atom (O), a nitrogen atom (N), or a sulfur atom (S), (2) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹, Z², or Z³ groups is not a hydrogen atom, (3) R is the same or different carbyl groups having between 1 and 3 carbon atoms, (4) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (5) the R^{a2-a4} may be linear or branched carbyl groups having between about 12 to 40 carbon atoms, (6) the R^{d,f,x} groups may be hydrogen atoms or linear or branched carbyl groups having between 1 and 10 carbon atoms, and (7) R^{g,h,i,w,z} are linear or branched carbyl groups having between 1 and 10 carbon atoms;
Z¹,Z²,Z³-Py-m-(RSH)₂ (IV)
where: (1) Py is pyridine, (2) -m- means the two RSH groups meta to each other, (3) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹, Z², and Z³ groups is not hydrogen, (4) R is the same or different carbyl groups having between 1 and 3 carbon atoms, (5) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (6) the R^{a2-a4} may be linear or branched carbyl groups having between about 12 to 40 carbon atoms, (7) the R^{d,f,x} groups may be hydrogen atoms or linear or branched carbyl groups having between 1 and 10 carbon atoms, and (8) R^{g,h,i,w,z} are linear or branched carbyl groups having between 1 and 10 carbon atoms; and
Z¹,Z²-Cp-m-(RSH)₂ (V)
where: (1) Cp is 5-member aromatic ring, where one of the carbon atoms of the Cp ring may be replaced by an oxygen atom, a nitrogen atom, or a sulfur atom, (2) -m- means that the two RSH groups are meta to each other or occupy the 2 and 5 positions of the five membered Cp ring, (3) Z¹ and Z² groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹ and Z² groups is not a hydrogen atom, (4) R is the same or different carbyl groups having between 1 and 3 carbon atoms, (5) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (6) the R^{a2-a4} may be linear or branched carbyl groups having between about 12 to 40 carbon atoms, (7) the R^{d,f,x} groups may be hydrogen atoms or linear or branched carbyl groups having between 1 and 10 carbon atoms, and (8) R^{g,h,i,w,z} are linear or branched carbyl groups having between 1 and 10 carbon atoms.

13. The composition of claim 12, wherein the absorbate or mixture thereof comprise compounds of the general formula (I):
Z¹,Z²,Z³-A(SH)ₖ(RSH)ₗ(CHₘ₁(SH)ₘ₂)ₙ (I)
where: (1) A is an aromatic group, where one of the carbon atoms of the group may be replaced by an oxygen atom (O), a nitrogen atom (N), or a sulfur atom (S), (2) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹, Z², or Z³ groups is not a hydrogen atom, (3) R is the same or different carbyl groups having between 1 and 3 carbon atoms, (4) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (5) the R^{a2-a4} may be linear or branched carbyl groups having between about 12 to 40 carbon atoms, (6) the R^{d,f,x} groups may be hydrogen atoms or linear or branched carbyl groups having between 1 and 10 carbon atoms, (7) R^{g,h,i,w,z} are linear or branched carbyl groups having between 1 and 10 carbon atoms, (8) k, l, m1, m2 and n are integers, (9) k has a value between 1 and 3, when l and n have a value of 0, (10) l has a value between 1 and 3, when k and n have a value of 0, (11) n has a value of 1, when k and l have a value of 0, and (12) m1+m2 is equal to 3. In certain embodiments, at least two of Z¹, Z², and Z³ groups are not hydrogen atoms. In other embodiments, all of the Z¹, Z², and Z³ groups are not hydrogen atoms.

14. The composition of claim 12, wherein the absorbate or mixture thereof comprise compounds of the general formula (II):
Z¹,Z²,Z³-ACHₘ₁(SH)ₘ₂ (II)
where: (1) A is an aromatic group, where one of the carbon atoms of the group may be replaced by an oxygen atom (O), a nitrogen atom (N), or a sulfur atom (S), (2) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{c}R^{f}, an amido group NR^{x}C(O)R^{y}, an oligoethylene glycol (OEG) group, or a polyethylene glycol (PEG) group provided that at least one of the Z¹, Z², or Z³ groups is not a hydrogen atom, (3) R is the same or different carbyl groups having between 1 and 3 carbon atoms, (4) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (5) the R^{a2-a4} may be linear or branched carbyl groups having between about 12 to 40 carbon atoms, (6) the R^{d,f,x} groups may be hydrogen atoms or linear or branched carbyl groups having between 1 and 10 carbon atoms, (7) R^{g,h,i,w,z} are linear or branched carbyl groups having between 1 and 10 carbon atoms, (8) m1 and m2 are integers, and (9) m1+m2 is equal to 3.

15. The composition of claim 12, wherein the absorbate or mixture thereof comprise compounds of the general formula (III):
Z¹,Z²,Z³-A-m-(RSH)₂ (III)
where: (1) A is an aromatic group, where one of the carbon atoms of the group may be replaced by an oxygen atom (O), a nitrogen atom (N), or a sulfur atom (S), (2) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹, Z², or Z³ groups is not a hydrogen atom, (3) R is the same or different carbyl groups having between 1 and 3 carbon atoms, (4) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (5) the R^{a2-a4} may be linear or branched carbyl groups having between about 12 to 40 carbon atoms, (6) the R^{d,f,x} groups may be hydrogen atoms or linear or branched carbyl groups having between 1 and 10 carbon atoms, and (7) R^{g,h,i,w,z} are linear or branched carbyl groups having between 1 and 10 carbon atoms.

16. The composition of claim 12, wherein the absorbate or mixture thereof comprise compounds of the general formula (IV):
Z¹,Z²,Z³-Py-m-(RSH)₂ (IV)
where: (1) Py is pyridine, (2) -m- means the two RSH groups meta to each other, (3) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹, Z², and Z³ groups is not hydrogen, (4) R is the same or different carbyl groups having between 1 and 3 carbon atoms, (5) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (6) the R^{a2-a4} may be linear or branched carbyl groups having between about 12 to 40 carbon atoms, (7) the R^{d,f,x} groups may be hydrogen atoms or linear or branched carbyl groups having between 1 and 10 carbon atoms, and (8) R^{g,h,i,w,z} are linear or branched carbyl groups having between 1 and 10 carbon atoms.

17. The composition of claim 12, wherein the absorbate or mixture thereof comprise compounds of the general formula (V):
Z¹,Z²-Cp-m-(RSH)₂ (V)
where: (1) Cp is 5-member aromatic ring, where one of the carbon atoms of the Cp ring may be replaced by an oxygen atom, a nitrogen atom, or a sulfur atom, (2) -m- means that the two RSH groups are meta to each other or occupy the 2 and 5 positions of the five membered Cp ring, (3) Z¹ and Z² groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹ and Z² groups is not a hydrogen atom, (4) R is the same or different carbyl groups having between 1 and 3 carbon atoms, (5) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (6) the R^{a2-a4} may be linear or branched carbyl groups having between about 12 to 40 carbon atoms, (7) the R^{d,f,x} groups may be hydrogen atoms or linear or branched carbyl groups having between 1 and 10 carbon atoms, and (8) R^{g,h,i,w,z} are linear or branched carbyl groups having between 1 and 10 carbon atoms.

18. The composition of claim 12, further comprises two or more compounds of formulas (I-V).

19. A substrate composition comprising:
a surface including a self-assembled monolayer composition formed thereon, there the self-3 assembled monolayer composition comprises an absorbate or mixture thereof comprise compounds of the general formulas (I-V):
Z¹,Z²,Z³-A(SH)ₖ(RSH)ₗ(CHₘ₁(SH)ₘ₂)ₙ (I)
where: (1) A is an aromatic group, where one of the carbon atoms of the group may be replaced by an oxygen atom (O), a nitrogen atom (N), or a sulfur atom (S), (2) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹, Z², or Z³ groups is not a hydrogen atom, (3) R is the same or different carbyl groups having between 1 and 3 carbon atoms, (4) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (5) the R^{a2-a4} may be linear or branched carbyl groups having between about 12 to 40 carbon atoms, (6) the R^{d,f,x} groups may be hydrogen atoms or linear or branched carbyl groups having between 1 and 10 carbon atoms, (7) R^{g,h,i,w,z} are linear or branched carbyl groups having between 1 and 10 carbon atoms, (8) k, l, m1, m2 and n are integers, (9) k has a value between 1 and 3, when l and n have a value of 0, (10) l has a value between 1 and 3, when k and n have a value of 0, (11) n has a value of 1, when k and I have a value of 0, and (12) m1+m2 is equal to 3. In certain embodiments, at least two of Z¹, Z², and Z³ groups are not hydrogen atoms. In other embodiments, all of the Z¹, Z², and Z³ groups are not hydrogen atoms;
Z¹,Z²,Z³-ACHₘ₁(SH)ₘ₂ (II)
where: (1) A is an aromatic group, where one of the carbon atoms of the group may be replaced by an oxygen atom (O), a nitrogen atom (N), or a sulfur atom (S), (2) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹, Z², or Z³ groups is not a hydrogen atom, (3) R is the same or different carbyl groups having between 1 and 3 carbon atoms, (4) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (5) the R^{a2-a4} may be linear or branched carbyl groups having between about 12 to 40 carbon atoms, (6) the R^{d,f,x} groups may be hydrogen atoms or linear or branched carbyl groups having between 1 and 10 carbon atoms, (7) R^{g,h,i,w,z} are linear or branched carbyl groups having between 1 and 10 carbon atoms, (8) m1 and m2 are integers, and (9) m1+m2 is equal to 3;
Z¹,Z²,Z³-A-m-(RSH)₂ (III)
where: (1) A is an aromatic group, where one of the carbon atoms of the group may be replaced by an oxygen atom (O), a nitrogen atom (N), or a sulfur atom (S), (2) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹, Z², or Z³ groups is not a hydrogen atom, (3) R is the same or different carbyl groups having between 1 and 3 carbon atoms, (4) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (5) the R^{a2-a4} may be linear or branched carbyl groups having between about 12 to 40 carbon atoms, (6) the R^{d,f,x} groups may be hydrogen atoms or linear or branched carbyl groups having between 1 and 10 carbon atoms, and (7) R^{g,h,i,w,z} are linear or branched carbyl groups having between 1 and 10 carbon atoms;
Z¹,Z²,Z³-Py-m-(RSH)₂ (IV)
where: (1) Py is pyridine, (2) -m- means the two RSH groups meta to each other, (3) Z¹, Z², and Z³ groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹, Z², and Z³ groups is not hydrogen, (4) R is the same or different carbyl groups having between 1 and 3 carbon atoms, (5) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (6) the R^{a2-a4} may be linear or branched carbyl groups having between about 12 to 40 carbon atoms, (7) the R^{d,f,x} groups may be hydrogen atoms or linear or branched carbyl groups having between 1 and 10 carbon atoms, and (8) R^{g,h,i,w,z} are linear or branched carbyl groups having between 1 and 10 carbon atoms; and
Z¹,Z²-Cp-m-(RSH)₂ (V)
where: (1) Cp is 5-member aromatic ring, where one of the carbon atoms of the Cp ring may be replaced by an oxygen atom, a nitrogen atom, or a sulfur atom, (2) -m- means that the two RSH groups are meta to each other or occupy the 2 and 5 positions of the five membered Cp ring, (3) Z¹ and Z² groups are the same or different and comprise a hydrogen atom, a carbyl group R^{a1}, halogenated carbyl group R^{a2}, a fluorinated carbyl group R^{a3}, a per-fluorinated carbyl group R^{a4}, an alkoxy group OR^{b} group, an amine group NR^{c}R^{d}, an amido group C(O)NR^{e}R^{f}, an amido group NR^{x}C(O)R^{y}, an oligoethylene glycol (OEG) group, or a polyethylene glycol (PEG) group provided that at least one of the Z¹ and Z² groups is not a hydrogen atom, (4) R is the same or different carbyl groups having between 1 and 3 carbon atoms, (5) the R^{a1} and the R^{c,e,y} groups may be linear or branched carbyl groups having between about 12 to 40 carbon atoms and sufficient hydrogens atoms to satisfy valencies, where one or more carbon atoms may be replaced by oxygen atoms, NR^{g} groups, C(O)NR^{h}Rⁱ groups, or NR^{w}C(O)R^{z} groups, (6) the R^{a2-a4} may be linear or branched carbyl groups having between about 12 to 40 carbon atoms, (7) the R^{d,f,x} groups may be hydrogen atoms or linear or branched carbyl groups having between 1 and 10 carbon atoms, and (8) R^{g,h,i,w,z} are linear or branched carbyl groups having between 1 and 10 carbon atoms.

20. The composition of claim 19, wherein the self-assembled monolayer composition further comprises two or more compounds of formulas (I-V).
